(19) United States
(12) Patent Application Publication
(10) Pub. No.: US 2025/0325648 A1
(43) Pub. Date: Oct. 23, 2025

(54) Dual-Action Recombinant Vesicular Stomatitis Virus (RVSV)-Based Vaccine (DAV) Against COVID-19 and Influenza Viruses

(71) Applicant: University of Manitoba, Winnipeg (CA)

(72) Inventors: Xiaojian Yao, Winnipeg (CA); Zhujun Ao, Winnipeg (CA)

(21) Appl. No.: 18/293,427

(22) PCT Filed: Jun. 28, 2022
(86) PCT No.: PCT/CA2022/051028

§ 371 Date: Jan. 30, 2024

Related U.S. Application Data
(60) Provisional application No. 63/236,455, filed on Aug. 24, 2021.

(51) Int. Cl. A61K 39/145; A61K 39/00; A61K 39/215; A61P 31/16; C07K 14/005; C12N 7/00

(52) U.S. Cl. CPC A61K 39/145; A61K 39/215; A61P 31/16; C07K 14/005; C12N 7/00; A61K 2039/5256; A61K 2039/70; C12N 2760/14122; C12N 2760/16122; C12N 2760/20222; C12N 2760/20243; C12N 2770/20022; C12N 2770/20034

(57) ABSTRACT

Described herein is a replicative Vesicular stomatitis virus (rVSV) comprising: a first Filoviridae glycoprotein comprising one or more influenza virus matrix 2 ectodomain peptide inserted into the first Filoviridae glycoprotein; and a second Filoviridae glycoprotein comprising a SARS-CoV2 Spike protein peptide inserted into the second Filoviridae glycoprotein, or a first Filoviridae glycoprotein comprising one or more influenza virus matrix 2 ectodomain peptide inserted into the first Filoviridae glycoprotein and a non-functional but immunogenic SARS-CoV2 Spike protein. The Spike protein or Spike protein peptide can be derived from different CoV-2 variants. The rVSV can be used as a Dual Action vaccine for vaccinating individuals simultaneously against both influenza virus and SARS CoV2 virus.

### PRIOR APPLICATION INFORMATION

The instant application claims the benefit of U.S. Provisional patent application Ser. No. 63/236,455, filed Aug. 24, 2021 and entitled “Dual-Action recombinant Vesicular Stomatitis Virus (rVSV)-based Vaccine (DAV) against COVID-19 and influenza viruses”, the entire contents of which are incorporated herein for all purposes.

### BACKGROUND OF THE INVENTION

The recent and ongoing outbreak of Coronavirus disease 2019 (COVID-19) has called for serious and urgent global attention (1, 24). The COVID-19 disease is caused by a newly emerged virus strain of Severe Acute Respiratory Syndrome (SARS) known as SARS-CoV-2 (24). Although the case fatality ratio (CFR) of COVID-19 can only be determined at the end of the outbreak, an estimated global CFR was calculated to be 3-4% in March 2020 shockingly more than the seasonal influenza outbreak (Z). While in August 2020, the infection fatality ratio was estimated by WHO to be 0.5-1% (32). Since the identification of the SARS-CoV-2 sequences (49), extensive efforts worldwide have been focused on developing effective vaccines and antiviral drugs against SARS-CoV-2. Up to now, there are several licensed vaccines which have been successfully developed for COVID-19 (21).

SARS-CoV-2 belongs to a betacoronavirus subfamily that includes enveloped, large and positive-stranded RNA viruses responsible for causing severe respiratory system, gastrointestinal and neurological symptoms (3, 19, 25, 50). The human coronavirus (CoV) was first identified in 1960 and constituted about 30% of the causes of the common cold. Among the identified human CoVs are NL63, 229E, OC43, HKU1, SARS-CoV, the Middle East respiratory syndrome (MERS)-CoV, and SARS-CoV-2 (36, 40). A recent study has revealed that SARS-CoV-2 was closely related (88% identity) to two SARS-like CoVs that were isolated from bats in 2018 in China, but it was less related to SARS-CoV (79%) and MERS-CoV (about 50%) (28). The key determinant for the infectivity of SARS-CoV-2 depends on the host specificity with the viral surface-located trimeric spike glycoprotein (SP), which is commonly cleaved by host proteases into an N-terminal S1 subunit and a membrane-embedded C-terminal S2 region (17). Recent studies revealed that an SP mutation, Aspartic acid (D) changed to Glycine (G) at amino acid position 614, in the S1 domain has been found in high frequency (65% to 70%) in April to May of 2020, that was associated with an increased viral load and significantly higher transmission rate in infected individuals, but no significant change with disease severity (22, 27). Subsequent studies also suggested that G614 SP mutant pseudotyped retroviruses infected ACE2-expressing cells markedly more efficiently than those with D614 SP (27). It has to be noted that a new Delta variant (B11.617.2) of SARS-CoV-2 was first found in India in December 2020. Only after several months, this particular variant spread to more than 98 countries around the world, becoming the dominant variant in many countries, including India, the U.K., Israel and the United States (12). Up to now, the Delta variant is the most contagious of all the known SARS-CoV-2 variants. Some research suggests that it's more than twice as transmissible as the original SARS-CoV2 strain. A recent study found that people infected by Delta variant had viral loads that can went up to 1,260 times higher than that of individuals infected with the original strain in 2020 (23). So, it is very necessary to develop some efficient ways to block the Delta variant transmission and infection.

Influenza virus disease is another contagious respiratory illness. Influenza virus has four types including Influenza A, B, C and D among which influenza A and B are of economic and medical importance to humans (9). Surprisingly, 100 years after a major pandemic infection caused by influenza virus A killed approximately 50 million people globally in 1918 (18, 31), influenza virus infection still poses a high threat to the health sector globally (43). According to the Centre for Disease Control (CDC), there are still pediatric deaths and young people deaths associated with different influenza infections (8). The fatality rate from influenza virus is not as high as that in previous years in the US; however, in developing countries and underdeveloped countries, there are still high levels of influenza infection, and consequently fear of emergence of new strain(s) of influenza virus. It should be noticed that the reduced numbers of influenza virus infection experienced currently is due to the availability of the vaccination each year, however, there are some issues regarding the production of vaccine based on predictions which may not be always be clear match with influenza infection in circulation in a given year. Also, this effort has not successfully eradicated the influenza virus infection (16, 45). Because of this, CDC recently emphasizes the need for a universal vaccine against influenza viral infection (16). Therefore, it is also urgent to develop a universal vaccine that must be used to elicit immune responses that can prevent or attenuate the infection of different strains of influenza virus.

As both COVID-19 and influenza are both contagious respiratory infections, which cause a wide range of illness from asymptomatic or mild through to severe disease and death; these respiratory infections continue to have dramatic impacts to challenge to public health, food systems and the world of work. Up to now, there are several licensed vaccines have been successfully developed for COVID-19 (20, 35), while the influenza vaccines have been available to get vaccinated each year to prevent influenza infection. Unfortunately, each of the prepared vaccines can only target either SARS-CoV2 or influenza, so, it is necessary to develop an universal vaccine which can simultaneously against both SARS-CoV2 and influenza viruses. Moreover, among the surface proteins of influenza virus is a conserved extracellular domain, Matrix-2 (M2), which has also been found promising in the development of a universal vaccine for influenza viral infection due to its highly conservation and stability (14, 39).

Vesicular stomatitis virus (VSV) is a single-stranded negative-sense RNA virus belong in the family Rhabdoviridae. Although VSV can cause illness in livestock and some animals, it is highly restricted to cause disease in humans by the human IFN response and generally does not cause any or only very mild symptoms (3). The VSV platform has been used as the attenuated replication-competent vaccine that induces a rapid and robust immune response to viral antigens after a single immunization and has been shown to protect against several pathogens (11_13, 29, 37, 41). Especially, the VSV-based Zaire Ebola glycoprotein vaccine (rVSV-ZEBOV) that expresses the EBOV GP has been considered safe and highly immunogenic and showed promising efficacy against EBOV in a phase III clinical trial (15, 41).

### SUMMARY OF THE INVENTION

According to a first aspect of the invention, there is provided a replicative Vesicular stomatitis virus (rVSV) comprising:
a first Filoviridae glycoprotein comprising one or more influenza virus matrix 2 ectodomain peptide inserted into the first Filoviridae glycoprotein; and
a second Filoviridae glycoprotein comprising a SARS-CoV2 Spike protein peptide inserted into the second Filoviridae glycoprotein.

According to another aspect of the invention, there is provided a method of targeting an influenza virus matrix 2 ectodomain peptide and a SARS-CoV2 Spike protein peptide to a dendritic cell comprising:
providing an rVSV as described above; and
immunizing an individual in need of immunization against the influenza virus or the SARS-CoV2 virus with an effective amount of the rVSV.

According to another aspect of the invention, there is provided use of rVSV as described above for targeting the influenza virus matrix 2 ectodomain peptide and the SARS CoV2 Spike protein peptide to a dendritic cell.

According to another aspect of the invention, there is provided a method of eliciting an immune response against an influenza virus matrix 2 ectodomain peptide and/or a SARS-CoV2 Spike protein peptide comprising:
providing a rVSV as described above and
immunizing an individual in need of immunization against influenza virus matrix 2 ectodomain peptide and/or SARS-CoV2 Spike protein peptide with an effective amount of the rVSV.

According to another aspect of the invention, there is provided a method of eliciting an immune response against an influenza virus and/or a SARS-CoV2 comprising:
providing a rVSV as described above and immunizing an individual in need of immunization against influenza virus and/or SARS-CoV2 with an effective amount of the rVSV.

### BRIEF DESCRIPTION OF THE DRAWINGS

FIG. 1. Construction and rescue of rVSV Delta SP and influenza M2e bivalent vaccines. A) Schematic diagram of the Delta SPΔC and EboGPΔM-tM2e immunogens present in the bivalent vaccines. a. SARS-CoV-2 Delta-SPΔC_{A742} (SPΔC1), containing a C-terminal 17 aa (DEDDSEPVLKGVKLHYT) deletion and a I742A mutation as indicated. The nine mutations in Delta SP are listed in lower part. b. Delta SPΔC2, containing the C-terminal 17 aa deletion and another 381 aa (encompassing aa744 to aa1124) deletion in S2 domain. The eight mutations in SPΔC2, are listed in lower part. c. EboGPΔM-RBD, the RBD of SARS-CoV-2 was used to replace the MLD domain in EboGP. d. EboGPΔM-tM2e, four copies of influenza virus M2 ectodomain (24 aa) polypeptide (tM2e) replaced the MLD domain in EboGP. B) The attenuated virus entry of SPΔC1. A549ACE2 cells were infected with equal amounts of SPΔC_{Delta}-PVs or SPΔC1-PVs (adjusted by P24) carrying Gaussia luciferase (Gluc) gene, as indicated. At 48 hrs after infection, the Gluc activity in the supernatant of different infected cultures was measured. Data represents Mean±SD of two replicates from a representative experiment out of three performed. C and D) The attenuated cell-to-cell fusion ability of SPΔC_{Delta}- or SPΔC1-mediated syncytia formation was analyzed by co-culturing the SPΔC_{Delta}- or SPΔC1-expressing 293T cells with A549ACE2 cells. The amounts of syncytia were counted after 24 hrs in 5 different views of microscope (C), and was also imaged under bright-field microscopy (D). E) Schematic diagram of VSV-EM2e/SPΔC1, VSV-EM2e/SPΔC2 and VSV-EM2e/ERBD and the virus rescuing procedures. 293T and Vero E6 co-culture cells were co-transfected with VSV-AG-EM2/SPΔC1, VSV-AG-EM2/SPAC or VSV-AG-EM2/RBD, and helping plasmids (T7, N, L, P plasmids). The supernatants containing V-EM2e/SPΔC1, V-EM2e/SPΔC2 and V-EM2e/ERBD viruses were used to infect Vero E6 cells to generate the rVSV stocks.

FIG. 2. Expression of V-EM2e/SPΔC1, V-EM2e/SPΔC2 or V-EM2e/ERBD in infected VeroE6 cells. A) The infection of V-EM2/SPΔC1, V-EM2/SPΔC2 or V-EM2/ERBD in Vero-E6 cells induced the cytopathic effects after four days of infection. B) Representative immunofluorescence images of Vero E6 cells infected with V-EM2e/SPΔC1, V-EM2e/SPΔC2, V-EM2e/ERBD or mock infected, stained with anti-SARS-CoV-2 RBD antibody (a-d) or anti-M2e antibody (i-l), and DAPI (e-h, m-p). C) VeroE6 cells infected with the rescued V-EM2/SPΔC1, V-EM2/SPAC or V-EM2/ERBD were lysed and processed with SDS-PAGE followed by WB with a rabbit anti-SARS-CoV-2 NTD antibody (top panel), a mouse antibody against influenza M2e (middle panel) or anti-VSV nucleocapsid (N) (low panel).

FIG. 3. Characterization of the replication kinetics and the cell tropisms of bivalent rVSV vaccine candidates. A) Each of bivalent VSV vaccine candidates or the rVSV expressing VSV-G (rVSV-wt) was used to infect different cell lines, including A549, MRC-5, U251 MG, CD4⁺ Jurkat T cells, human monocyte-derived macrophages (MDMs) and Dendritic cells (MDDCs). Supernatants were collected at different time points post infection as indicated and were titrated on Vero E6 cells. Data represent Mean±SD and were obtained from two replicates of a representative experiment out of two performed. B) The ability of induced cytopathic effects in A549, U251 MG and CD4⁺ Jurkat T cells, by each rVSV were observed after 4 days of infection under microscopy. a, V-EM2e/ERBD; b, V-EM2e/SPΔC1; c, V-EM2e/SPΔC2, and d. rVSVwt.

FIG. 4. Anti-SARS-CoV-2 RBD and anti-influenza M2e immune responses induced by immunization with different bivalent VSV vaccine candidates. A) Schematic of the bivalent rVSV vaccine candidate immunization protocol in mouse. BALB/c mice were immunized with V-EM2e/SPΔC1, V-EM2e/SPΔC2 or V-EM2e/ERBD via intramuscular (IM) or intranasal (IN) routes, as indicated. The mice sera were collected at day 13 and 28 and were measured for anti-SARS-CoV-2 RBD IgG and IgA antibody levels (B-D) or measured for anti-M2e IgG and IgA antibody levels (F-H). E, D) The anti-SARS-CoV-2 RBD and anti-M2e IgA antibody levels at 28 days. Data represent Mean±SD. Statistical significance was determined using unpaired T-test. *, P<0.05; **, P<0.01; ***, P<0.001; ****, P<0.0001.

FIG. 5. rVSV Delta SP vaccine candidates elicited neutralization antibodies. The neutralization titers (50% inhibition) in immunized mice sera against SpΔC_{WT}-Luc-PVs (A), SpΔC_{Delta}-Luc-PVs (B), SpΔC_{beta′}-Luc-PVs (D), SpΔC_{b.1.617}-Luc-PVs (E), and SpΔC_{Omic}-Luc-PVs (F) infections. VSV-G-Luc-PVPs (C) was used as negative control. The mouse serum of each immunization group collected at day 28 were pooled together, 2× serially diluted and incubated with different Luc-PVs (˜10⁴ RLU). Then, the mixtures were added in A549_{ACE2}.cell cultures and the infection of PVs was determined by Luciferase assay at 48-66 hrs post infection. The percentage of infection was calculated compared with no serum control and neutralizing titers were calculated by using sigmoid 4PL interpolation with GraphPad Prism 9.0, as described in Materials and Methods. Data represent Mean±SD and were obtained from over three independent experiments. Statistical significance was determined using ordinary one-way ANOVA test and Turkey's test. *, P<0.05; **, P<0.01; ***, P<0.001; ****, P<0.0001.

FIG. 6. T-cell cytokine response induced by bivalent VSV vaccine candidates. Splenocytes isolated from immunized mice (as described in FIG. 4A) were cultured without peptide (no-peptide control, NC) (A-E), or stimulated with SARS-CoV-2 SP subunit 1 (S1) peptide pool (F-J) or influenza M2e peptide (K-O) (1 μg/mL for each peptide). After 4 days of stimulation, supernatants were collected, and the release of Th1 (IFN-γ, TNF-α) and Th2 (IL-4, IL-5, IL-13) cytokines in the supernatants was quantified with an MSD U-plex mouse cytokine immunoassay kit and counted in the MESO Quickplex S0120 instrument. Each symbol indicated one individual mouse. Statistical significance between the two groups was determined using an unpaired t test. *, P<0.05; **, P<0.01; ***, P<0.001; ****, P<0.0001.

FIG. 7. Mice immunized with V-EM2/SPΔC1 were protected against the lethal challenge of H1N1 and H3N2 influenza viruses. A) Schematic of the bivalent VSV vaccine candidate immunization and influenza virus challenge protocol used in the study. For H1N1 challenge experiment, the BALB/c mice were immunized with 1×10⁸ TCID₅₀ (IM) or 1×10⁵ TCID₅₀ (IN) of V-EM2e/SPΔC1 or PBS at day 0 and day 14. On day 27, the blood samples were collected and measured for anti-influenza M2e antibody level by ELISA (B). At day 28, all the mice were challenged with 2100 PFU of H1N1 influenza virus. Weight loss (C) and survive rates (D) of the mice were monitored daily for 2 weeks. E) Viral loads in the lung tissue of immunized mice and PBS group at day 5 post H1N1 challenge were measured in MDCK cell line, as described in Materials and Methods. For H3N2 challenge experiment, the BALB/c mice were immunized with 1×10⁵ TCID₅₀ (IN) of V-EM2e/SPΔC1 or PBS at day 0 (single-dose, SD), and at day 0 and 14 (double-dose, DD). At day 28, all the mice were challenged with 1.4×10⁴ PFU of H3N2. F) Weight loss; G) Survive rates; H) Viral loads in the lung tissue of immunized mice and PBS group at day 6 after H3N2 challenge.

FIG. 8. V-EM2/SPΔC1 and V-EM2/SPΔC2 provided protection against SARS-CoV-2 Delta infection in Syrian Hamsters. A) Schematic of the bivalent VSV vaccine candidate immunization and SARS-CoV-2 Delta variant challenge protocol used in the study. B) Total serum anti-SARS-CoV-2 spike IgG titers in hamsters following prime and boost vaccination. C) Weight loss in the vaccine immunized or the control Syrian hamsters following infection with SARS-CoV-2 Delta variant. D) Viral RNA levels in oral swabs on day 3 following infection with SARS-CoV-2 Delta variant. E) Infectious SARS-CoV-2 Delta virus titers in nasal turbinates and lungs tissues on day 5 following infection with SARS-CoV-2 delta. n=10 for B (each time point), n=10 for C, 10 through day 28 and 10 at day 42), n=10 for D (at day 3 post-infection) and n=5 for E (from day 5 post-infection). Statistical significance assessed by two-way analysis of variance with multiple comparisons (A), mixed effects analysis with multiple comparisons (B), Krusal-Wallis test with multiple comparisons (C and D). *=p<0.05, **=p<0.01, ***=p<0.001, ****=p<0.0001. For B, colored asterisks indicate significant differences between the same colored group compared with the PBS group. Shown are medians for each group in A, C, and D, and mean+SEM in B.

FIG. 9. Immunization of rVSVΔG-EboGPΔM-M2e/EboGPΔM-RBD in mice induced anti-SARS-CoV-2 SP antibody response. Balb/c mice were immunized with rVSVΔG-EboGPΔM-M2e/EboGPΔM-RBD through intramuscular (1×10⁷ TCID₅₀/mouse) or intranasal (1×10⁵ TCID₅₀/mouse) routes, as indicated. After 14 days of immunization, the sera from mice were collected, diluted as 1:50 and 1:100, measured for anti-SARS-CoV-2 SP antibody response determined by ELISA coated with SARS-CoV-2 RBD peptide.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Published PCT Application WO 2019/113688, incorporated herein by reference for its teachings regarding the EboGP expression system, describes a series of Ebolavirus envelope glycoprotein (EboGP)-based and Marburgvirus envelope glycoprotein (MarvGP)-based chimeric fusion proteins that are still able to maintain an efficient EboGP-mediated virus entry in various cell types including human antigen-presenting cells (APCs) and macrophages, while presenting large polypeptides at the apex and the sides of each EboGP monomer.

As discussed therein, the mucin-like domain is generally accepted as encompassing residues 305 or 308 to 501 of the EboGP peptide sequence and amino acid residues 257-501 of the Marburg virus. For example, the deletion of 178 amino acids within the mucin-like domain permits the insertion of larger peptides. That is, deletion of these 178 amino acids and replacement thereof with an antigenic peptide of interest results in the peptide of interest being presented or displayed or expressed at the apex and sides of the glycoprotein monomer. This is an example of what is referred to therein and herein as “tolerated deletions”, that is, deletions of amino acids within the mucin-like domain that do not significantly impair presentation or display of the inserted peptide at the apex and sides of the fusion glycoprotein. Other suitable tolerated deletions will be apparent to one of skill in the art and/or can be confirmed or determined using routine experimentation. For example, in some embodiments, the deletion is from 305 to 483 of the Ebola glycoprotein.

EboGP can be efficiently incorporated into retroviral particles resulting in significantly facilitated cell entry in human DCs and macrophages, and stimulating significantly higher immune responses. Previously, it was known that the MLD domain or a tolerated deletion thereof could be replaced by heterologous peptide in order to target peptides to antigen-presenting cells, but it was not known if inserted peptides could be targeted specifically to dendritic cells. As discussed herein, targeting to dendritic cells is critical for generating an immune response against a peptide that has traditionally generated a poor immune response.

As discussed herein, we have designed and generated different attenuated replicating VSV simultaneously expressing EboGPΔM-M2e fusion proteins and SARS-CoV-2 Spike protein peptide fusion proteins. In some embodiments, the Spike protein peptide fusion proteins are EboGPΔM-RBD fusion proteins, SARS-CoV2 Delta variant SPΔCa742 and Delta variant SPΔS2ΔC proteins, referred to herein as :rVSV-EboGPΔM-M2e/EboGPΔM-RBD, rVSV-EboGPΔM-M2e/SPΔCa742 and rVSV-EboGPΔM-M2e/SPΔS2ΔC respectively. As will be appreciated by one of skill in the art, other SARS CoV2 peptides, preferably highly conserved Spike CoV2 protein peptides and/or immunogenic Spike CoV2 protein peptides, that is, Spike CoV2 protein peptides that will elicit an immune response may be used within the invention. It is further noted that, as discussed herein, while some Spike protein peptides from the SARS CoV2 Delta variant are used in some examples, Spike protein peptides from other variants, particularly variants of interest and/or emergent SARS-CoV2 viruses, may be used within the invention.

As discussed herein, any suitable rVSV construct with an influenza virus protein, preferably the influenza virus matrix 2 ectodomain peptide, and a SARS CoV2 Spike protein peptide act as a Dual-Action VSV-based Vaccines (DAV) against SARS-CoV2 (including Delta variant) and influenza virus infections. As discussed below, these rVSV constructs have a promising safety profile because of the use of live-attenuated VSV vaccine (15, 41). Furthermore, the strong Dendritic cell (DC) targeting ability of the EboGPΔM (2, 4) makes these rVSV constructs strong vaccines, as discussed herein.

In this invention, we have generated an attenuated replicating recombinant Vesicular Stomatitis Virus (VSV)-based dual-Action Vaccine that is able to express both the SARS-CoV2 Spike glycoprotein (SP) or its component(s) and an influenza M2 ectodomain (M2e) which is fused with a DC-targeting/activation domain (EboGPΔM), derived from a Zaire Ebola glycoprotein. Immunization with this vaccine will be able to induce robust host immune responses that can protect from severe SARS-CoV2 Delta variants and influenza virus infections

### The Unique Features are at Least:

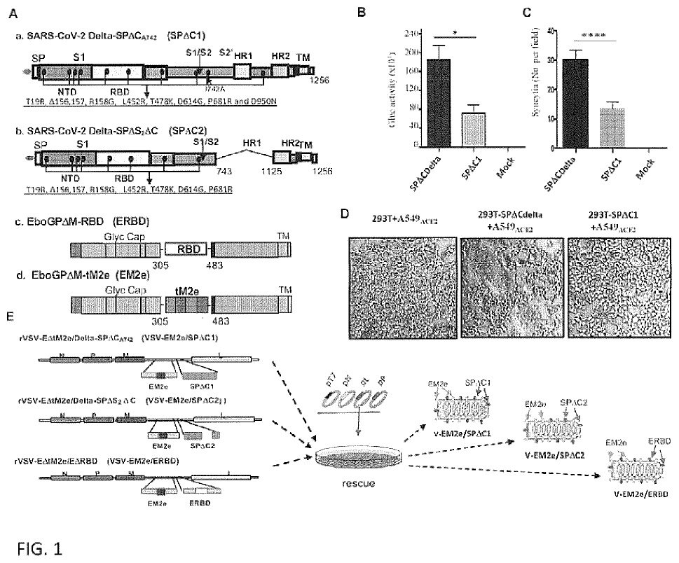

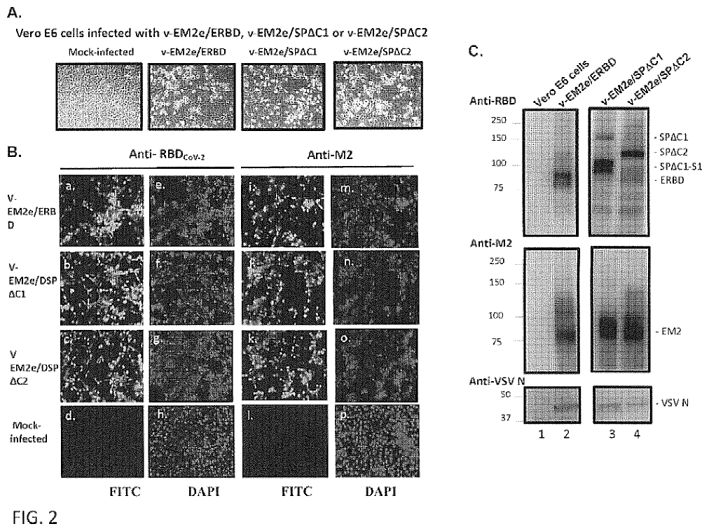

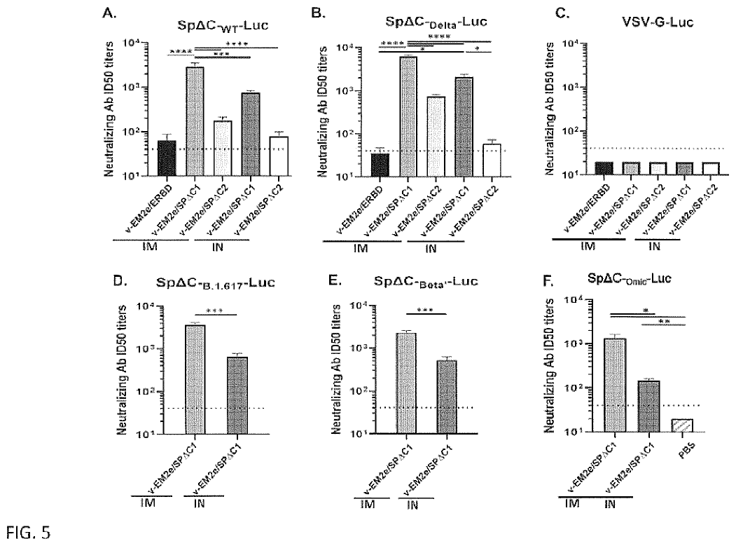

1) In some embodiments, the (VSV)-based dual-Action Vaccine simultaneously expresses both a SARS-CoV2 Spike protein and at least one, for example, two or more or in some embodiments four copies of highly conserved ectodomain of influenza virus M2 protein in a vector. As a result of this arrangement, the vaccine is able to elicit sufficient host immune responses to be protective against both SARS-CoV2 infection (including Delta variant) and various influenza virus infections. 2) Also described herein is the use of special components of SARS-CoV2 SP as antigen in rVSV vaccine: including 1) use of the receptor binding domain (RBD) of SARS-CoV2 SP which is fused with a DC-targeting/activation domain (EboGPΔM) (FIG. 1); or 2) Use of an S2-deleted SARS-CoV2 SP (SP-ΔS2ΔC) which is non-functional but presents large parts of the SARS-CoV-2 SP to the host immune system (As will be appreciated by one of skill in the art, while the Delta variant is used in the Examples, a similar construct can be derived from any SARS-CoV-2 strain, for example, a circulating variant or a variant of concern, as discussed herein), 3) Use of SP-ΔS2ΔC, which comprises a C-terminal 17 amino acid (aa) deletion (FIGS. 2 and 5) that enhances SARS-CoV2 SP expression, for example, on the surface of infected cells as well as its incorporation into produced virus particles which will in turn increase its exposure to the host immune system, and 4) Use of any SARS-CoV2 SP peptide in a non-functional form as discussed herein. One illustrative example of a suitable non-functional form of SP may be for example a virus entry/maturation defective SARS-CoV2 SPAC.

Specifically, because VSV is a replicating virus, it is desirable in some embodiments to use a non-functional whole Spike protein, such as a maturation-defective or attenuated form to make it non-functional. That is, as will be appreciated by one of skill in the art, we can use whole and/or non-functional spike protein peptides, as discussed herein.
3) As discussed herein, in some embodiments, a SARS-CoV2 Delta variant SP is used (FIG. 2) to specifically target Delta variant transmission and infection. However, as discussed herein, the rVSV can be modified by replacing the SPAC with any other emergent and/or highly transmissible/pathogenic SARS-CoV2 SP variants, so as to provide broader and more efficient protection against particular, specific SARS-CoV2 variants, such as for example, circulating SARS-CoV2 strains or SARS-CoV2 strains of interest or concern.
4) As discussed herein, in some embodiments of the invention, we have used our novel DC-targeting vaccine technology by fusing a DC-targeting/activation domain (EboGPΔM), derived from Ebola GP with a four-copies of conserved M2 ectodomain (M2e) of Influenza A from human, Avian, and swine to generate a recombinant VSV-based vaccine system.

The 1st advantage of this fusion technology is that in this rVSV vaccine platform, we do not need to use VSV glycoprotein (VSVG) for rVSV replication which in turn will avoid potential risks in vivo. We have already shown that this EboGPΔM-based fusion protein, including EboGPΔM-M2e, has a strong ability to enter into various cells including the host antigen presenting cells, such as for example dendritic cells and macrophages (6, 47). This strong DC-targeting ability of EboGPΔM significantly enhances the immunogenicity of rVSV expressed antigens (4, 6, 48).

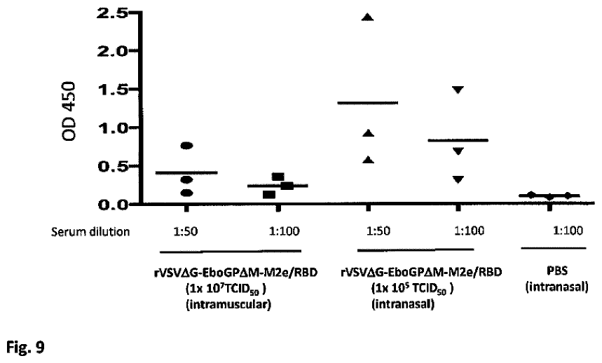

The 2nd advantage is that the EboGPΔM is able to hold a large polypeptides (up to 241 amino acids) without affecting its cell targeting and entry ability (6, 48). In some embodiments, we have inserted SARS-CoV-2 receptor-binding domain (RBD, 193aa) into the EboGPΔM (FIG. 1), and inserted into rVSV vector. The resulted rVSV is able to replicate and express EboGPΔM-RBD (FIG. 2), and induce anti-SARS immune response (shown in FIG. 9).

According to an aspect of the invention, there is provided a replicative Vesicular stomatitis virus (rVSV) comprising:
a first Filoviridae glycoprotein comprising one or more influenza virus matrix 2 ectodomain peptide inserted into the first Filoviridae glycoprotein; and
a second Filoviridae glycoprotein comprising a SARS-CoV2 Spike protein peptide inserted into the second Filoviridae glycoprotein.

In some embodiments of the invention, the one or more influenza virus matrix 2 ectodomain peptide is inserted into the first Filoviridae glycoprotein in frame such that the one or more influenza virus matrix 2 ectodomain peptide is expressed as a fusion protein with the first Filoviridae glycoprotein.

In some embodiments of the invention, the SARS-CoV2 Spike protein peptide is inserted into the second Filoviridae glycoprotein in frame such that the SAR CoV-2 Spike protein peptide is expressed as a fusion protein with the second Filoviridae glycoprotein.

In some embodiments, the SARS CoV2 Spike protein peptide comprises 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more or 25 or more consecutive amino acids of the SARS CoV2 Spike protein sequence. That is, as will be appreciated by one of skill in the art, “Spike protein peptide” indicates that a peptide that is derived from the Spike protein and is not necessarily a full-length protein but is preferably a peptide that is immunogenic, that is, that is sufficient to induce an immune response, is used.

As discussed herein, SARS CoV2 Spike protein, as used herein, refers to the amino acid sequence of the SARS CoV2 original strain Spike protein sequence as well as any of the known variants thereof.

In some embodiments, the mucin-like domain comprises amino acids 305-501 of the Ebola Virus glycoprotein.

In some embodiments, the mucin-like domain consists of amino acids 305-501 of the Ebola virus glycoprotein.

In some embodiments, the mucin-like domain comprises amino acids 257-425 of Marburg virus glycoprotein.

In some embodiments, the mucin-like domain consists of amino acids 257-425 of Marburg virus glycoprotein.

In some embodiments, the mucin-like domain is a tolerated deletion of the mucin-like domain. That is, in some embodiments, the peptide or protein of interest, that is, the influenza virus matrix 2 ectodomain peptide and/or the SARS-CoV2 Spike protein peptide is not only inserted in frame into the mucin-like domain of the Filoviridae glycoprotein, the peptide or protein of interest also replaces at least some of the mucin-like domain. That is, as discussed below, the peptide or protein of interest is inserted in frame into a tolerated deletion of the mucin-like domain, as discussed herein.

In some embodiments of the invention, the tolerated deletion is amino acids 305-501 or 305-483 of the Ebola glycoprotein. However, as discussed herein and as will be apparent to one of skill in the art, other tolerated deletions of the mucin-like domain may be used within the invention.

In some embodiments of the invention, the first Filoviridae glycoprotein is Ebola glycoprotein.

In some embodiments of the invention, the first Filoviridae glycoprotein is a tolerated deletion of the mucin-like domain of the Ebola glycoprotein.

In some embodiments of the invention, the one or more influenza virus matrix 2 ectodomain peptide is inserted in frame in the tolerated deletion of the mucin-like domain of the first Ebola glycoprotein.

In some embodiments of the invention, the influenza virus matrix 2 ectodomain peptide comprises at least 23 consecutive amino acids of the influenza virus matrix 2 ectodomain peptide.

In some embodiments of the invention, the one or more influenza virus matrix 2 ectodomain peptide is selected from: a human influenza virus; an avian influenza virus; a swine influenza virus and combinations thereof.

In some embodiments of the invention, there are two or more influenza virus matrix 2 ectodomain peptides inserted in frame in the tolerated deletion of the mucin-like domain of the first Ebola glycoprotein.

In some embodiments of the invention, each respective one influenza virus matrix 2 ectodomain peptide is separated from a respective adjacent influenza virus matrix 2 ectodomain peptide by a spacer.

In some embodiments of the invention, there are four influenza virus matrix 2 ectodomain peptides inserted in frame in the tolerated deletion of the mucin-like domain of the first Ebola glycoprotein.

In some embodiments of the invention, the four influenza virus matrix 2 ectodomain peptides are two human influenza virus matrix 2 ectodomain peptides, one avian matrix 2 ectodomain peptide and one swine matrix 2 ectodomain peptide.

In some embodiments of the invention, a cassette comprising the four influenza virus matrix 2 ectodomain peptides comprises the amino acid sequence as set forth in SEQ ID NO:6.

As will be apparent to one of skill in the art and as discussed herein, the use of the expression “cassette” is intentional and is used specifically to indicate the ease with which the matrix 2 ectodomain peptide construct in one embodiment of the invention can be substituted for a different matrix 2 ectodomain peptide construct.

In some embodiments of the invention, each respective one influenza virus matrix 2 ectodomain peptide is separated from a respective adjacent influenza virus matrix 2 ectodomain peptide by a spacer.

In some embodiments, there is provided a virus-like particle comprising derived from the rVSV described above.

As known to those of skill in the art, the 24 aa M2 ectodomain peptide is very conserved in different species of influenza viruses. As an example, the matrix 2 ectodomain peptides from human influenza virus, avian influenza virus and swine influenza virus are produced below:

(human, SEQ ID NO: 1)
SLLTEVETPIRNEWGCRQNDSSD;
(avian. SEQ ID NO: 2)
SLLTEVETPTRNGWECKCSDSSD;
(human(SEQ ID NO: 3)
SLLTEVETPIRNEWGCRCNDSSD;
and
(swine(SEQ ID NO: 4)
SLLTEVETPIRNGWECRCNDSSD.

As will be appreciated by one of skill in the art, this can be used to generate a consensus sequence as set forth below:

(SEQ ID NO: 5)
SLLTEVETP(I/T)RN(G/E)W(G/E)C(R/K)C(N/S)DSSD.

As discussed herein, in some embodiments of the invention, 2 or more copies of the matrix 2 ectodomain peptide are used in the glycoprotein fusion protein. In these embodiments, the respective matrix 2 ectodomain peptides are separated from each other by a spacer peptide. As will be appreciated by one of skill in the art, any suitable spacer known in the art which allows for the respective domains to be presented separately and individually may be used within the invention. In some embodiments of the invention, the first and last matrix 2 ectodomain peptide in sequence are separated from the glycoprotein or tolerated deletion thereof as discussed herein by a suitable spacer.

As used herein, as will be apparent to those of skill in the art, “spacer” refers to non-native peptide sequence that is positioned between two different, for example, non-contiguous peptide sequences. Specifically, the spacer or linker is provided so that the two different peptide sequences are capable of or are arranged to fold independently. In some embodiments, the spacer is preferably selected so that the spacer acts as a flexible linking sequence between the two peptides. Examples of suitable spacers are provided herein; however, other suitable spacers will be readily apparent to one of skill in the art and are within the scope of the invention.

In some embodiments, the spacer is selected from the group consisting of: GGG, GGGS, GSA, GPGPG and combinations thereof.

In some embodiments, the spacer is GGG.

In some embodiments of the invention, the fusion protein comprises:

(SEQ ID NO: 6)
^{GGG}SLLTEVETPIRNEWGCRCNDSSD^{GGG}SLLTEVETPTRNGWECKCSDSS
D^{GGG}SLLTEVETPIRNEWGCRCNDSSD^{GGG}SLLTEVETPIRNGWECRCNDS
SD^{GGG}.

As discussed below, in one example provided below, a construct comprising two human, one avian, and one swine M2 peptide was designed. As will be apparent to those of skill in the art, this construct provides protection not only against human influenza viruses but also from avian or swine influenza viruses which have been known to jump species. Furthermore, because of the high degree of conservation between matrix domains from other influenza virus species, this construct will provide broad range protection against many influenza virus strains. While two copies of M2e from human virus is used in this example to increase antigenicity of M2e from human virus, in some embodiments, it may not be necessary to use 4 Matrix2 domain peptides. For example, a construct may have three matrix 2 ectodomain peptides: one each of human, avian and swine influenza virus matrix 2 ectodomain peptides for example, or may have 2, 5, 6 or more different Matrix2 peptides.

Surprisingly however, it was found that only one copy of M2e (FIG. 9) induced a much weaker immune response to M2e, indicating that more than one copy is needed in some embodiments.

In some embodiments of the invention, the Filoviridae virus glycoprotein is the Ebola virus glycoprotein and four copies of the matrix 2 ectodomain peptide are inserted in a tolerated deletion of the mucin-like domain spanning amino acids 305-483 of the native Ebola virus glycoprotein:

(SEQ ID NO: 7)
MGVTGILQLPRDRFKRTSFFLWVIILFQRTFSIPLGVIHNSTLQVSDVD
KLVCRDKLSSTNQLRSVGLNLEGNGVATDVPSATKRWGFRSGVPPKVVN
YEAGEWAENCYNLEIKKPDGSECLPAAPDGIRGFPRCRYVHKVSGTGPC
AGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLE
SRFTPQFLLQLNETIYTSGKRSNTTGKLIWKVNPEIDTTIGEWAFWETK
KNLTRKIRSEEGPGGGSLLTEVETPIRNEWGCRCNDSSDGGGSLLTEVE
TPTRNGWECKCSDSSDGGGSLLTEVETPIRNEWGCRCNDSSDGGGSLLT
EVETPIRNGWECRCNDSSDGGGSRNTIAGVAGLITGGRRTRREAIVNAQ
PKCNPNLHYWTTQDEGAAIGLAWIPYFGPAAEGIYIEGLMHNQDGLICG
LRQLANETTQALQLFLRATTELRTFSILNRKAIDFLLQRWGGTCH

In some embodiments of the invention, the second Filoviridae glycoprotein is Ebola glycoprotein.

In some embodiments of the invention, the second Filoviridae glycoprotein is a tolerated deletion of the mucin domain of the Ebola glycoprotein.

In some embodiments of the invention, the SARS-CoV2 Spike protein peptide is inserted in frame in the tolerated deletion of the mucin-like domain of the second Ebola glycoprotein.

In some embodiments of the invention, the SARS-CoV2 Spike protein peptide is selected from the group consisting of: a Spike protein RBD domain peptide; Spike protein peptide SPΔCa742 or Spike protein peptide SPΔS2ΔC.

In some embodiments of the invention, the SARS-CoV2 Spike protein peptide is the Spike protein RBD domain.

As will be appreciated by one of skill in the art and as discussed herein, RBD domain from any suitable SARS-CoV2 strain may be used within the invention. One example of the RBD domain peptide sequence is provided below:

(SEQ ID NO: 8)
PNITNLCPFGEVFNATRFASVYAWNRKRISNCVADYSVLYNSASFSTFK
CYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAG
STPCNGVEGFNCYFPLQSYGFQPTNGVGYQPYRVVVLSFELLHAPATV

In some embodiments of the invention, the Filoviridae virus glycoprotein is the Ebola virus glycoprotein and the RBD domain is inserted in a tolerated deletion of the mucin-like domain spanning amino acids 305-483 of the native Ebola virus glycoprotein:

(SEQ ID NO: 9)
MGVTGILQLPRDRFKRTSFFLWVIILFQRTFSIPLGVIHNSTLQVSDVD
KLVCRDKLSSTNQLRSVGLNLEGNGVATDVPSATKRWGFRSGVPPKVVN
YEAGEWAENCYNLEIKKPDGSECLPAAPDGIRGFPRCRYVHKVSGTGPC
AGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLE
SRFTPQFLLQLNETIYTSGKRSNTTGKLIWKVNPEIDTTIGEWAFWETK
KNLTRKIRSEEGGPNITNLCPFGEVFNATRFASVYAWNRKRISNCVADY
SVLYNSASFSTFKCYGVSPTKLNDLCFTNVYADSFVIRGDEVRQIAPGQ
TGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVV
LSFELLHAPSRNTIAGVAGLITGGRRTRREAIVNAQPKCNPNLHYWTTQ
DEGAAIGLAWIPYFGPAAEGIYIEGLMHNQDGLICGLRQLANETTQALQ
LFLRATTELRTFSILNRKAIDFLLQRWGGTCHILGPDCCIEPHDWTKNI
TDKIDQIIHDFVDKTLPDQGDNDNWWTGWRQWIPAGIGVTGVIIAVIAL
FCICKFVF

The SARS-CoV-2 SP DELTA variant includes the following mutations: T19R, Δ156,157, R158G, L452R, T478K, D614G, P681R and D950N.

In some embodiments of the invention, the Spike protein peptide SPΔCa742 is SARS-CoV2 Delta variant Spike protein peptide SPΔCa742.

Specifically, in these embodiments, DEDDSEPVLKGVKLHYT (SEQ ID NO:12) is deleted. Specifically, in some embodiments, this region of the C-terminus is deleted to increase the amount of Spike protein moved to the cell surface and incorporated into virus particles. This in turn will result in the Spike protein being more efficiently exposed to the host immune system. However, without this deletion, the Spike protein will be more localized to the cytoplasm of the cells, which will be effective as well, albeit in a different manner.

In some embodiments of the invention, the Spike protein peptide SARS-CoV2 Delta variant Spike protein peptide SPΔCa742 comprises the amino acid sequence:

(SEQ ID NO: 10)
MFVFLVLLPLVSSQCVNLDTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESXXGVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYRFRLFRKSNLKPFERDISTEIYQAGSKPC
NGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQGVNCTEVPVAI
HADQLTPTWRVYSTGSNVFQTRAGCLIGAEHVNNSYECDIPIGAGICA
SYQTQTNSRRRARSVASQSIIAYTMSLGAENSVAYSNNSIAIPTNFTI
SVTTEILPVSMTKTSVDCTMYACGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRFNGIGVT
QNVLYENQKLIANQFNSAIGKIQDSLSSTASALGKLQNVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKF

[symbol]

[symbol]

In some embodiments, the Spike protein peptide SPΔS2ΔC is SARS-CoV2 Delta variant Spike protein peptide SPΔS2ΔC

In some embodiments, the Spike protein peptide SPΔS2ΔC comprises the amino acid sequence:

(SEQ ID NO: 11)
MFVFLVLLPLVSSQCVNLDTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESXXGVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYRFRLFRKSNLKPFERDISTEIYQAGSKPC
NGVEGENCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQGVNCTEVPVAI
HADQLTPTWRVYSTGSNVFQTRAGCLIGAEHVNNSYECDIPIGAGICA
SYQTQTNSRRRARSVASQSIIAYTMSLGAENSVAYSNNSIAIPTNFTI
SVTTEILPVSMTKTSVDCTMYAC-------381aa deleted-----
------------------------------------------------
NCDVVIGIVNNTVYDPLQPELDSFKEELDKYFKNHTSPDVDLGDISGI
NASVVNIQKEIDRLNEVAKNLNESLIDLQELGKYEQYIKWPWYIWLGF
IAGLIAIVMVTIMLCCMTSCCSCLKGCCSCGSCCKF

[symbol]

As discussed above for the matrix 2 peptide, the RBD domain peptide may be considered to be inserted in the tolerated deletion of the Ebola glycoprotein as a cassette. Similarly, Spike protein peptide SPΔCa742 and Spike protein peptide SPΔS2ΔC may also be considered as being inserted into the tolerated deletion of the glycoprotein so that these peptides can be replaced with corresponding peptides from other SARS-CoV2 virus strains, as discussed herein.

As discussed herein, the rVSV comprises a first fusion glycoprotein and a second fusion glycoprotein downstream of the first fusion glycoprotein, that is, 3′, to the first fusion glycoprotein. As a result of this arrangement, the two fusion glycoproteins are produced from the rVSV in approximately identical amounts. As will be appreciated by one of skill in the art, this overcomes many of the difficulties associated with simultaneous or sequential vaccination with two different vaccines, as discussed herein. In some embodiments, there is provided a nucleic acid encoding the rVSV described above.

In some embodiments of the invention, the rVSV further comprises at least VSV N, P, M and L genes.

According to another aspect of the invention, there is provided a method of targeting influenza virus matrix 2 ectodomain peptide and a SARS-CoV2 Spike protein peptide to a dendritic cell comprising:
providing an rVSV as described above
administering to an individual an effective amount of the rVSV.

According to another aspect of the invention, there is provided use of rVSV as described above for targeting the influenza virus matrix 2 ectodomain peptide and the SARS CoV2 RBD to a dendritic cell.

According to another aspect of the invention, there is provided a method of eliciting an immune response against an influenza virus matrix 2 ectodomain peptide and/or a SARS-CoV2 Spike protein peptide comprising:
providing a rVSV as described above and
immunizing an individual in need of immunization against influenza virus matrix 2 ectodomain peptide and/or SARS-CoV2 Spike protein peptide with an effective amount of the rVSV.

According to another aspect of the invention, there is provided a method of eliciting an immune response against an influenza virus and/or a SARS-CoV2 comprising:
providing a rVSV as described above and
immunizing an individual in need of immunization against influenza virus and/or SARS-CoV2 with an effective amount of the rVSV.

As will be appreciated by one of skill in the art, the immune response may be in an individual, in particular, an individual in need of immunization against influenza virus, wherein the individual may be a human.

As will be appreciated by one of skill in the art, an individual in need of such treatment may be an individual who is at risk of being exposed to the influenza virus or who is in a high-risk group as defined by the WHO and/or an individual who gets the annual flu shot, for example, pregnant women, children 5 years of age and younger, the elderly, health care workers and people who have chronic illnesses or are immunocompromised.

Alternatively, an individual in need of such treatment may be an individual who is at risk of being exposed to the SARS-CoV2 virus or who is in a high risk group as defined by the WHO, for example, an older adult, especially over 60 years of age, or of any age with a chronic medical condition selected from the group consisting of: lung disease; heart disease; high blood pressure; diabetes; kidney disease; liver disease; dementia; and stroke; and any individual who is immunocompromised, including those with an underlying medical condition, such as cancer or taking medications which lower the immune system, such as chemotherapy, or are living with obesity (BMI of 40 or higher)

Furthermore, as discussed herein, the immune response may be increased or enhanced compared to the immune response obtained from immunizing an individual of similar age and general condition with the matrix 2 ectodomain peptide or the SARS-CoV2 Spike protein peptide either alone or in combination without insertion in the mucin-like domain.

As will be appreciated by one of skill in the art, the immune response generated by the rVSV comprising a first fusion protein of Filoviridae glycoprotein and influenza virus matrix 2 ectodomain peptide and a second fusion protein of Filoviridae glycoprotein and SARS-CoV2 Spike protein peptide may be increased or enhanced over the immune response that would be generated in a control individual, that is, an individual of similar age or condition as the immunized individual, immunized with the influenza virus matrix 2 ectodomain peptide and/or with the SARS-CoV2 Spike protein peptide alone, that is, separately or simultaneously by mixing two different vaccine preparations, as discussed herein.

As will be appreciated by one of skill in the art, as used herein, “an effective amount” of the rVSV comprising the fusion peptides is an amount that is sufficient to elicit an immune response. Such an effective amount will depend on several factors, for example, the age, weight and general condition of the individual. Methods for determining such an effective amount will be readily apparent to one of skill in the art and/or easily determined through routine experimentation.

As will be apparent to one of skill in the art, while two vaccine products could be mixed together for vaccination, this effectively at least doubles the amount of work for all procedures including vaccine production, the safety test, and the efficacy test. Analysis of the results will also be more complicated because of possible variations in administration and/or vaccine efficacy in different subjects.

As discussed herein, we have generated an attenuated replicating rVSV vaccine products that are able to target against SARS-CoV2 and influenza viruses. This Dual-Action rVSV vaccine simultaneously expresses an EboGPΔM-M2e fusion protein and an EboGPΔM-SARS CoV2 Spike protein peptide fusion protein. While in some embodiments, the EboGPΔM-SARS CoV2 Spike protein peptide fusion protein or SARS CoV2 Spike protein peptides are selected from the group consisting of EboGP-RBD, SARS-CoV2 DeltaSPΔCa742 and DeltaSPΔS2ΔC, other suitable Spike protein peptides may be used in the invention and are within the scope of the invention.

The immunization of individuals, for example, human subjects or patients, with these rVSV vaccines induces sufficient immune response to immunize these individuals against SARS-CoV2 and influenza viruses, as discussed below.

Also, our study revealed that the replicating kinetics of these rVSV-EboGPΔM-M2e/EboGPΔM-RBD viruses were significantly slower than the wild type VSV, suggesting an attenuated feature of rVSV-EboGPΔM-M2e/EboGPΔM-RBD virus. One of the reasons for its attenuation could be the lack of VSV-G glycoprotein in the rVSV.

New SARS-CoV-2 variants from UK (B.1.1.7), South Africa (B.1.351), and Brazil (P.1) have quickly spread across the world and become predominant. Some recurrent mutations in SP are shared by these variants and may contribute to the increased transmission and re-infection, and potentially reduced sensitivity to antibodies. Some recurrent mutations in SP are shared by these variants and may contribute to the increased transmission and re-infection, and reduced sensitivity to host immune responses (17, 19, 29, 38). Three RBD mutations (K417N/T, E484K, N501Y) are shared by B.1.351 and P.1 (13, 44).

It was reported that, in the December 2020, a new SARS-CoV-2 Delta variant (B11.617.2) was identified in India. After several months, this specific SARS-CoV-2 variant spread to over 98 countries, becoming the dominant variant in more than a dozen of those countries, including India, the U.K., Israel and the United States (12). To date, the Delta variant is the most contagious of all the known SARS-CoV-2 variants, possibly more than twice as transmissible as the original strain of the coronavirus with viral loads up to 1,260 times higher than that of individuals infected with the original strain in 2020 (23). It is known that there are nine mutations existing within Delta variant SP that may result in at least partial escape from current immune protection.

COVID-19 and influenza are both highly contagious respiratory diseases with a wide range of severe symptoms and cause great disease burdens globally. Especially, the ongoing pandemic of COVID-19 has been the most serious threat to global public health [1, 24]. Thus, it has become urgent to develop a bivalent vaccine that can be able to target simultaneously these two contagious respiratory diseases. In this study, we have developed rVSV bivalent vaccines, including V-EM2/SPΔC1 and V-EM2/SPΔC2, that specifically target SARS-CoV-2 Delta variant and influenza viruses. Replication studies revealed that the replication kinetics of these rVSV viruses was much slower in all cell lines tested and they were unable to infect CD4+T lymphocytes, as compared to wild-type VSV, suggesting the highly attenuated characteristics of these rVSV-based vaccines. Importantly, the immunization studies in animal models have shown that the V-EM2e/SPΔC1- and V-EM2e/SPΔC2 are able to induce high humoral and cellular immune responses including the neutralizing antibodies against different SARS-CoV-2 SP-PV infections. Meanwhile, our studies have demonstrated that the immunization of V-EM2/SPΔC1 or V-EM2/SPΔC2 effectively protected hamsters or mice against the challenges of SARS-CoV-2 Delta variant and lethal H1N1 and H3N2 influenza viruses.

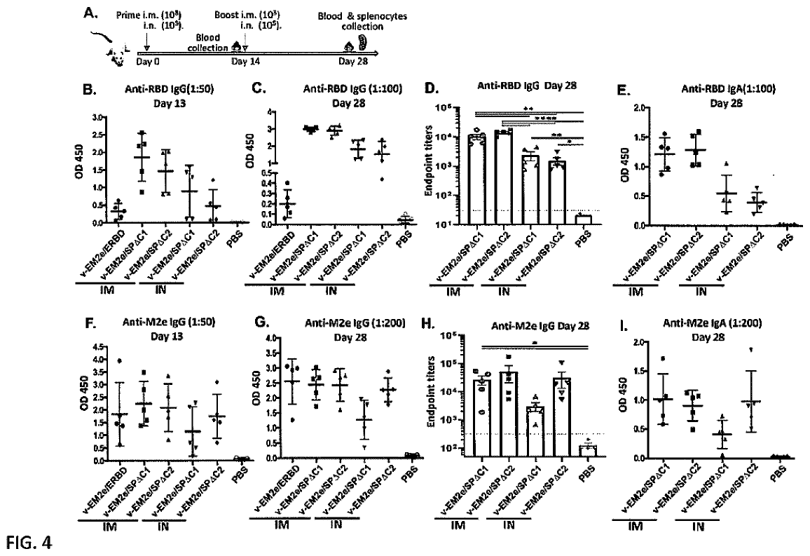

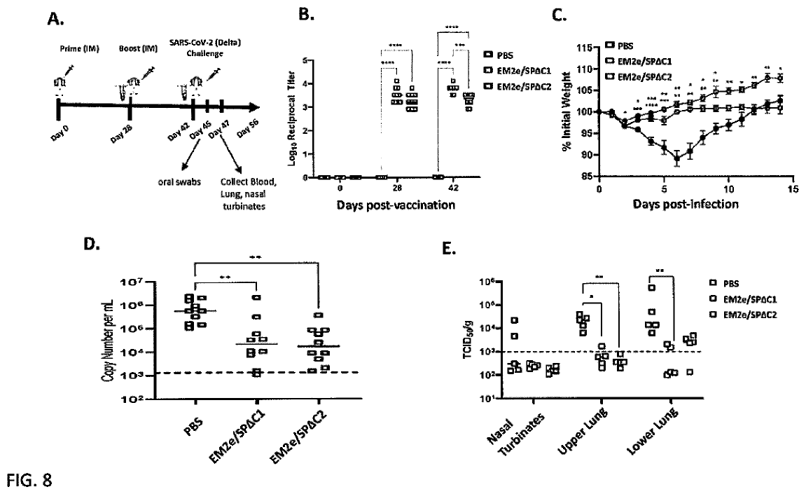

The SARS-CoV-2 Delta variant emerged in 2021 and quickly became the predominant circulating variant worldwide, showing increased potential for transmission and increased disease severity in humans [65]. The Delta variant also showed a significant immune evasion when comparing the protection of vaccine- or infection-elicited humoral immune responses against different variants [51-53]. This was due to amino acid changes in key residues of the viral spike protein, and indicated that variant-specific vaccine formulations could provide improved neutralization in immunized individuals. Therefore, an rVSV-based bivalent vaccine specifically targeting SARS-CoV-2 Delta variant has been generated in this study. The rVSV vaccine has been shown to be an ideal vaccine platform [54]. In addition to its safety and easy and scalable production, the rVSV vaccine is able to induce a rapid and robust immune response to viral antigens after a single immunization and has been used to protect against several pathogens [13, 37, 11, 29, 41], including SARS-CoV-2 [54, 55, 56, 57, 58]. In our study, we have demonstrated that the rVSV-based vaccines expressing either the full length SP or S1 domain derived from Delta variant provided efficient protection against the challenge of SARS-CoV-2 Delta variant (FIG. 8). This is agreement with the report by Malherbe, D. C et al showing a S1-expressed VSV protected against COVID-19 virus infection [55]. However, our data revealed that the immune response induced by V-EM2e/SPΔC2 expressing S1 tended to be weaker than that of V-EM2e/SPΔC1 expressing the full-length SPDelta (FIGS. 4 and 8). In line with this, the challenge experiment showed that although both vaccines protected infected hamsters from significant weight loss, in comparison with PBS animals losing more than 10% of their initial weight on average, the S1 vaccinated animals remained close to their initial weight during the recovery but no further weight gaining, indicating that there may have been other disease manifestations in these animals. However, they were significantly protected from disease during the acute stage of infection (FIG. 8). One explanation for the difference in immune response and protection induced by EM2e/SPΔC1 and V-EM2e/SPΔC2 could be that the S2 domain of SP may play an as yet undefined role in enhancing the induction of neutralizing antibodies. Indeed, a previous report indicated that two segments, 884-891 and 1116-1123, located in SP-S2, are very effective in inducing host immune responses [69]. Another interesting finding from our animal study was that the second dose of vaccine in hamsters did not significantly boost antibody titers, only showing a marginal benefit, at least in terms of the anti-SARS-CoV-2 SP antibody response (FIG. 8B), suggesting a single dose of VSV vaccination is sufficient to induce robust protective immune responses.

During COVID-19 pandemic, the continuing emergence of viral variants of concern (VOC) has resulted in a decrease in vaccine effectiveness in terms of preventing infection, likely due to reductions in neutralization capacity against specific variants, waning antibody responses over time, and relatively little mucosal antibody presented following vaccination [70-72]. Particularly, the new VOC of SARS-CoV-2 Omicron (B.1.1.529) designated in November of 2022 possesses an excessive number of mutations compared to other variants, especially with 32 amino acid residue changes in the SP [66]. Recent reports have shown that the rapid spread of this VOC was facilitated by its strong ability to re-infect pre-immunized individuals through its efficient transmissibility and high immune escape ability [67, 76]. Therefore, it is necessary to assess whether EM2e/SPΔC1 vaccine could induce a broad neutralization capacity against various variant's infection. Results from our study indicated that the V-EM2e/SPΔC1 vaccination in mice elicited high titers of neutralizing antibodies specifically against Delta SpΔC-PVs as well as SpΔC_{WT}-, SpΔC_{B.1.617}- and SpΔC_{Beta′}-PV infections, while exhibited partially inhibitory activity against SpΔC_{Omic}-PV infection (FIG. 5). Although the NAb titers against SpΔC_{Omic}-PV could still reach a level (>10³) that was likely comparable to the anti-2019-nCoV NAb titers detected in COVID-19-infected patients in a previous study [77], we still do not know at this moment whether our leading vaccine candidate V-EM2e/SPΔC1 could provide in vivo effective protection from Omicron variant infection, which requires further investigation.

It is important for vaccines to significantly reduce the potential for viral shedding and transmission in the respiratory system, therefore to limit disease outbreaks and break the transmission chains. Interestingly, in our study, either EM2e/SPΔC1 or V-EM2e/SPΔC2 was able to significantly reduce viral shedding as measured by viral RNA load in oral swabs on day 3 p.i., which is approximately the peak of viral replication and shedding [73]. Also, viral titers were reduced in both vaccinating groups compared with PBS animals in the upper lobes of lung, as well as in the lower lung in the full-length SP vaccination group. Additionally, while infectious virus was only detected in the nasal turbinates of two animals in the PBS group, no infectious virus was detected in any vaccinated animals in the nasal turbinates. This provides strong evidence that vaccination with either vaccine candidate via IM route can inhibit viral replication in the tissues of the upper and lower airways. Overall, our data suggest that each vaccine candidate provides significant protection in the Syrian hamster models against clinical disease, viral shedding, and viral replication, and that further development of this vaccine platform is warranted.

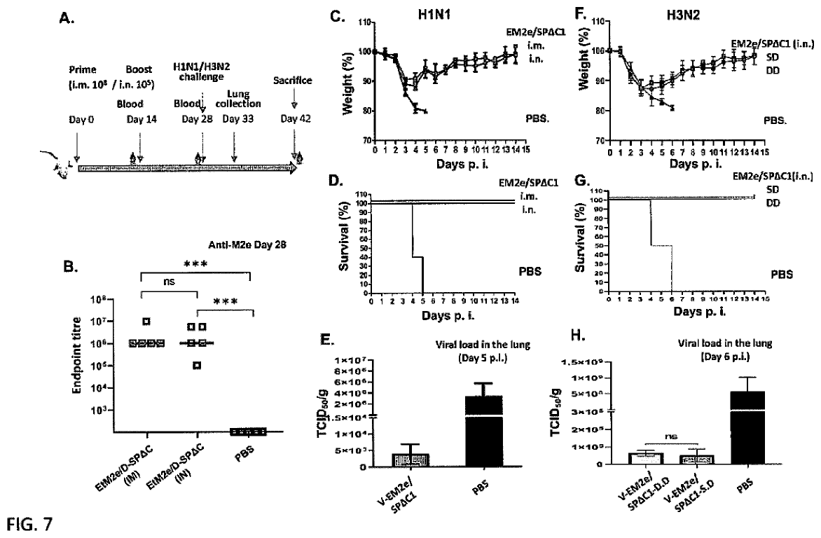

Given that COVID-19 and influenza both are contagious respiratory diseases, it is ideal and efficient for a vaccine to 1) provide simultaneous protection against infections of both SARS-CoV-2 and influenza viruses; and 2) to provide an intranasal vaccination strategy as a convenient and easy alternative to injection, which can trigger mucosal immunity in the nasal cavity. In our study, the bivalent vaccine has been designed to specifically target the SP of SARS-CoV-2 Delta strain and a highly conserved ectodomain of M2 (M2e) of influenza A viruses. The four copies of M2e domains from human, avian and swine virus strains [26](FIG. 1A, d) were used to induce broad heterosubtypic immune responses to influenza Type A viruses. Importantly, the bivalent vaccine candidate described in this study elicited a high level of M2e-specific immune responses (FIG. 4). Also, the lead vaccine candidate V-EM2/SPΔC1 via both IM or IN routes effectively protected mice from lethal H1N1 influenza virus infection (FIG. 8D-F), which confirmed our previous findings [68]. Furthermore, the results also revealed that even a single IN immunization with V-EM2/SPΔC1 achieved equally efficient protection from H3N2 challenge as compared to prime-boost IN immunization (FIG. 7G-1). It should be noted that the dose of VSV vaccine used for IN administration was 1000-fold lower than that for IM administration, indicating that the lower dose of rVSV vaccination through IN route may achieve equally sufficient immune responses and protection against influenza respiratory diseases. However, it remains to be investigated whether intranasal vaccination with V-EM2/SPΔC1 could also provide protection against infection with the SARS-CoV-2 Delta variant. Interestingly, a recent report indicated that intranasal vaccination of a VSV-SARS-CoV-2 resulted in protection in hamsters within 10 days prior to SARS-CoV-2 challenge, and animals did not show signs of pneumonia [37].

The rapid T cell response following vaccination is generally considered a key part of the immune response required to elicit effective protection. In this study, we observed strong T cell responses such as high levels of secreted cytokines, including IFN-γ, TNF-α, IL-4, IL-5 and IL-13, in splenocytes from mice stimulated with rVSV-based vaccine candidates. These findings implied that rVSV-based vaccines could elicit both humoral and cellular responses in mice. It is well known that the Th1 and Th2 cells are the major part of T cell immunity, wherein Th1 cells secrete Th1 cytokines and are responsible for the activation of B cells (producing IgG2a), macrophages, and NK/cytotoxic T cells, and Th2 cells mostly activate B cells (producing IgG1). Furthermore, Th1 cytokines, such as IFN-γ and TNF-α, tend to induce pro-inflammatory reactions, whereas Th2 cytokines, such as IL-4, IL-5 and IL-13, play anti-inflammatory roles to suppress excessive inflammation. To further investigate the cellular response of immunized mice, we analyzed the ratios of Th1/Th2 cytokines (IFN-γ/IL-5) and found a significant difference between the samples isolated from the IM- and IN-immunized mice, suggesting that IM immunization triggered a Th1-biased but relatively Th1/Th2-balanced cellular response, consistent with previous reported COVID-19 vaccines [74, 75], while IN immunization induced an extremely Th1-biased cellular response. Interestingly, a recent paper revealed that IL-13 plays a protective role in vitro to reduce SARS-CoV-2 replication, and cell-to-cell transmission [76]. In our results, only IM immunization showed high stimulation of IL-13, indicating a better potential to protect from SARS-CoV-2. Given the facts that the vaccination doses through IM and IN routes were different and the cytokine levels in respiratory tract were not measured in this study, the role of Th1 and Th2 responses via different delivery routes on the protective efficacy of the vaccine is still elusive. However, compared with more invasive IM immunization, the IN immunization process more closely resembles natural infection with SARS-CoV-2. Hence, more detailed studies are required to determine whether IN administration will enable the vaccine to establish more effective protection.

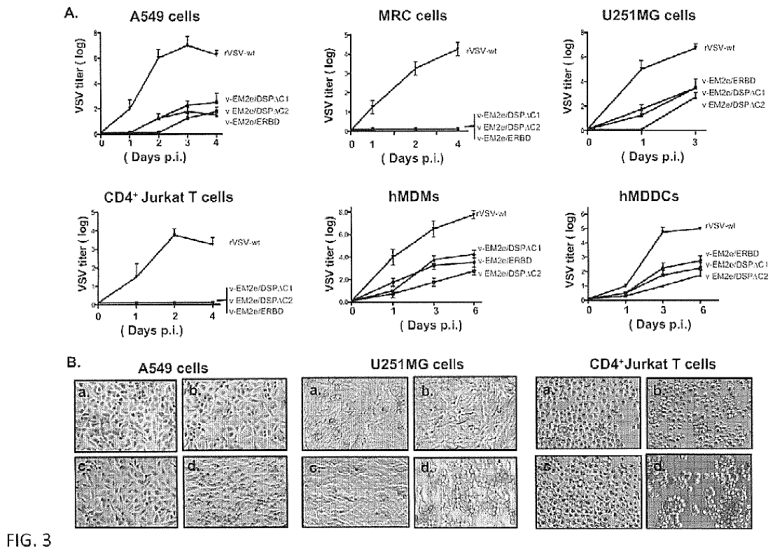

The safety profile is also an important issue for vaccine development. Even though the pathogenicity of the rVSVΔG vector is significantly attenuated compared to the wild-type VSV, the replacement of VSV-G with EM2 and SARS-CoV-2 SP affected the cell tropism of vaccine candidates. As expected, we observed much attenuated replication kinetics of V-EM2/SPΔC1 and V-EM2/SPΔC2 in various cell lines, including A549, a type II pulmonary epithelial cell line and MRC-5, a human lung fibroblast cell line, compared to rVSV expressing VSV-G. Except for Vero-E6 cells, these vaccines showed no, or much milder, cytopathic effects in most tested cell lines compared with VSV_{wt}, which induced significant cytopathic effects (FIG. 3). Importantly, the V-EM2/SPΔC1 and V-EM2/SPΔC2 do not target CD4⁺ T cells, which is also essential for protecting the immune system from attack. Given that SARS-CoV-2 SPDelta was able to efficiently mediate cytopathic effect (cell fusion) and subsequently cause cell death [59, 77], we introduced a mutation (I742A) into the SPΔC_{Delta} gene of V-EM2e/SPΔC1 to reduce SPDelta'S cytotoxicity. Indeed, the I742A mutation significantly reduced infectivity of a SPΔC_{Delta}-pseudovirus and its induced syncytia formation (FIG. 1B-D).

Overall, in this study, we have generated rVSV-based bivalent vaccines and demonstrated that immunization with these rVSV-vectored vaccines in hamster and/or mice induced strong protective immune responses, including neutralizing antibodies and/or cell-mediated immune responses against SARS-CoV2 Delta variants and influenza A viruses. Also, our study has demonstrated that immunization with the vaccine candidate V-EM2e/SPΔC1 effectively protected mice from lethal H1N1 and H3N2 influenza virus infections, and the IM immunization of V-EM2e/SPΔC1 and V-EM2e/SPΔC2 effectively protected hamsters against the challenges of SARS-CoV-2 Delta variant. Altogether, these studies provided substantial evidence for the high efficacy of this bivalent vaccine platform that can be used and easily adapted to produce new vaccines simultaneously against both emerging SARS-CoV-2 and influenza contagious respiratory infections.

To rapidly response to this critical situation, we have developed two vaccine candidates that can directly target this Delta variant (B1.617.2) by inserting genes encoding DeltaSPΔCa742, and DeltaSPΔS2ΔC respectively into rVSV (FIG. 2), as discussed herein. As discussed herein, this Dual-Action replicating rVSV vaccine can be easily modified by replacing DeltaSPΔCa742, or DeltaSPΔS2ΔC encoding gene sequence with other newly emergent SARS-CoV-2 SP from these variants to create new anti-COVID-19 rVSV vaccine products.

In this vaccine platform, we have used our novel DC-targeting vaccine technology by fusing a DC-targeting/activation domain (EboGPΔM), derived from EboGP with a four-copies of conserved M2 ectodomain (M2e) of Influenza A from human, Avian, and swine (48) (FIG. 1A). The big advantage of using EboGPΔM-based fusion protein is that this fusion protein is able to replace VSVG glycoprotein, and can act as the main glycoprotein for rVSV to efficiently enter into the host antigen presenting cells such as dendritic cells and macrophages (6, 48). This is also important to enhance the immunogenicity of introduced various antigens in the rVSV (4) and while avoiding more severe pathogenicity possibly induced by VSVG encoded rVSV vaccines, as discussed herein.

This is possible because the EboGPΔM was shown to be able to fuse various large heterologous polypeptides (up to 200 amino acids) (48).

As described herein, EboGPΔM is fused with a four-copies of highly conserved ectodomain of M2 (M2e) of influenza virus. The ectodomain of M2 has strong sequence conservation across all influenza A virus, and has been found promising in the development of a universal vaccine for influenza viral infection due to its stability and high conservation (10, 42, 44). Indeed, our study revealed that the rVSV-EboGPΔM-M2e can protect mice from H1N1 and H3N2 virus challenges.

As discussed herein, we have also prepared a number of Spike protein peptide fusion constructs.

In one embodiment, SARS-CoV-2 RBD was inserted into EboGPΔM as discussed herein, and the EboGPΔM-RBD encoding gene was placed 3′ to EboGPΔM-M2e within the rVSV vector. The data has demonstrated that the resulting rVSVΔG-EboGPΔM-M2e/EboGPΔM-RBD was able to express both EboGPΔM-RBD and EboGPΔM-M2e (FIG. 2). Furthermore, this vaccine candidate induced strong anti-SARS-CoV-2 S1 immune responses, especially administrated through intranasal route (FIG. 9).

In summary, this invention presents several rVSV-based dual-Action Vaccine products that can simultaneously express both a SARS-CoV2 SP or RBD, for example, from the Delta variant although any SARS-CoV strain may be used, and the highly conserved ectodomain of influenza virus M2 protein in a rVSV vector. As a result of this arrangement, the vaccine is able to elicit sufficient host immune responses that could not only prevent both SARS-CoV2 Delta variant and other variant infections, but also block various influenza infections.

As discussed herein, we do not need to use VSV glycoprotein (VSVG) for rVSV to enter and replicate in host cells. The EboGPΔM-based fusion protein has strong affinity to enter into the host cells, including antigen presenting cells such as dendritic cells and macrophages and therefore to significantly enhances the immunogenicity of rVSV expressed antigens (4, 50). Furthermore, the EboGPΔM is able to accept fusion with large polypeptides (up to 200 amino acids) (50). Beside the M2e of influenza virus as presented in the invention, EboGPΔM can also be fused with other large polypeptides, such as a influenza HA conserved polypeptide or an receptor-binding domain (193aa) of SARS-CoV2 SP in the recombinant VSV vector (shown in FIG. 1).

In another embodiment, a Dual-Action rVSV vaccine simultaneously expresses the SARS-CoV2 DeltaSPΔC or DeltaSPΔS2ΔC and an EboGPΔM-M2e fusion protein. The results showed that both DeltaSPΔC and an EboGPΔM-M2e were expressed during the infection in veroE6 cells.

Our study also revealed that the replicating kinetics of all of rVSV-EboGPΔM-M2e/EboGPΔM-RBD, rVSV-EboGPΔM-M2e/DeltaSPΔCa742, and rVSV-EboGPΔM-M2e/DeltaSPΔS2ΔC viruses were significantly slower than the wildtype VSV, suggesting an attenuated feature of these rVSV viruses. One of the reasons for its attenuation could be the lack of VSV-G glycoprotein in the rVSV. This is important because the VSV-G glycoprotein in a rVSV vector can be too efficient, making it a reluctant choice for commercialization.

In another embodiment of the invention, the full length DeltaSPΔCa742 protein, or an S2-deleted DeltaSPΔS2ΔC protein was inserted in the vector, and their expression confirmed (FIG. 2), as discussed above.

As discussed herein, this clearly demonstrates that the vaccine platform can be easily modified by replacing the respective inserts like “cassettes”, for example replacing SPΔCa742, or SPΔS2ΔC from the Delta variant used in one embodiment of the invention with gene(s) encoding for newly emergent SARS-CoV-2 SP variants to create new anti-COVID-19 rVSV vaccine products.

The invention will now be further explained and/or elucidated by way of examples; however, the invention is not necessarily limited to or by the examples.

### Example 1—Generation of rVSV Vector Encodes Genes Encoded for Both EboGPΔM-M2e and EboGPΔM-RBD

To develop VSV-based vaccine against both SARSCoV-2 and influenza, we first used PCR techniques to generate genes encode for amino acid sequences of M2e and SARSCoV-2 RBD. Then the RBD and M2 genes were inserted into EboGPΔM (6). In the EboGPΔM-M2e fusion protein, there are four copies of M2e polypeptide (M2e), including two copies of the highly conserved M2 ectodomain (24 aa) from human influenza (red), one copy of avian influenza M2 ectodomain (24 aa, green) and one copy of the swine influenza M2 ectodomain (26). The reason for using EboGPΔM-M2e is that our recent study has revealed that expression of a fusion protein EboGPΔM-M2e in rVSV was able to protect mice from H1N1 and H3N2 virus challenges (FIG. 2). These findings indicate the great potential of rVSV-EboGPΔM-M2e as an anti-influenza vaccine approach. By using the same fusion strategy, the RBD was fusion in frame into EboGPΔM to generate EboGPΔM-RBD (FIG. 1) Furthermore, the genes that encode for EboGPΔM-M2e and EboGPΔM-RBD were respectively inserted into a rVSV vector to position where VSV-G gene sequence located (FIG. 1). The attenuated replicating rVSV expressing both EboGPΔM-M2e and EboGPΔM-RBD was rescued in VeroE6 cells via a reverse genetics technology (4). After the rVSVΔG-EboGPΔM-M2e/EboGPΔM-RBD was successfully rescued, the rVSVΔG-EboGPΔM-M2e/EboGPΔM-RBD can replicate in VeroE6 cells and induce the cytopathic effect (FIG. 2).

### Example 2. Expression of Both EboGPΔM-RBD and EboGPΔM-M2e in rVSVΔG-EboGPΔM-M2e/EboGPΔM-RBD in the Infected VeroE6 Cells

To verify the expression of EboGPΔM-RBD and EboGPΔM-tM2e, we infected VeroE6 cells with rVSVΔG-EboGPΔM-M2e/EboGPΔM-RBD. Meanwhile the non-infected VeroE6 cells were used as control. After two days of infection, we collected the infected cells and non-infected cells. The presence of EboGPΔM-RBD and EboGPΔM-M2e were detected by indirect immunofluorescence assay with a rabbit anti-SARS-CoV-2 RBD antibody or anti-influenza M2 antibody. Results revealed the presence of EboGPΔM-RBD and EboGPΔM-M2e in the infected cells, but not in non-infected cells (FIG. 2).

Meanwhile, the expression of EboGPΔM-RBD and EboGPΔM-M2e were detected by SDS-PAGE and Western blot (WB) with a rabbit anti-SARS-CoV-2 RBD antibody or anti-influenza M2 antibody. The data showed both EboGPΔM-RBD and EboGPΔM-M2e were detected in rVSVΔG-EboGPΔM-M2e/EboGPΔM-RBD-infected cells, but not in non-infected VeroE6 cells (FIG. 2). All of these results demonstrate the specific expression of EboGPΔM-RBD and EboGPΔM-M2e in rVSVΔG-EboGPΔM-M2e/EboGPΔM-RBD-infected VeroE6 cells (FIG. 2). Also, the VSV nucleocapsid (N) protein was detected in the rVSVΔG-EboGPΔM-M2e/EboGPΔM-RBD-infected VeroE6 cells (FIG. 2).

### Example 3. Immunization of rVSVΔG-EboGPΔM-M2e/EboGPΔM-RBD in Mice Induced Anti-SARS-CoV-2 SP Antibody Response

Next, we tested whether Anti-SARS-CoV-2 SP immune responses can be induced by vaccination by rVSVΔG-EboGPΔM-M2e/EboGPΔM-RBD in mice. Balb/c mice were intramuscularly or intranasally immunized with rVSVΔG-EboGPΔM-M2e/EboGPΔM-RBD or PBS and after 14 days of immunization, the sera from mice were collected and assessed anti-SARS-CoV-2 SP antibody response determined by ELISA coated with SARS-CoV-2 RBD peptide. Results showed that the anti-SARS-CoV-2 SP specific humoral immune responses were detected in mice immunized with rVSVΔG-EboGPΔM-M2e/EboGPΔM-RBD (FIG. 9). Interestingly, our results revealed that the intranasal administration of rVSVΔG-EboGPΔM-M2e/EboGPΔM-RBD induced significantly higher antibody response than that of intramuscular administration (FIG. 9). Collectively, these results indicate that the expression of EboGPΔM-RBD in mice induced specific humoral antibodies against SARS-CoV-2 SP, especially through intranasal administration.

### Example 4. Generation of rVSV Vector Encodes Genes Encoding for Both EboGPΔM-M2e and SARS-CoV-2 DeltaSPΔCa742 or SARS-CoV-2 DeltaSPΔS2ΔC

Given the fact that a new SARS-CoV-2 Delta variant (B11.617.2), which is highly transmissible variant and spread to over 98 countries around the world, becoming the dominant variant in many countries, including India, the U.K., Israel and the United States within several months (12), we have generated two rVSV vaccine candidates, rVSV-EboGPΔM-M2e/SPΔCa742 and rVSV-EboGPΔM-M2e/SPΔS2ΔC, that express the SARS-CoV-2 Delta variant SP DeltaSPΔCa742 or DeltaSPΔS2ΔC, respectively in rVSV (FIG. 2). The detailed amino acid sequences of DeltaSPΔCa742 or SARS-CoV-2 DeltaSPΔS2ΔC are provided above. The reason for deleting 17aa at the C-terminus of DeltaSPΔCa742 or SARS-CoV-2 DeltaSPΔS2ΔC is based the fact that previous studies have revealed that the cytoplasmic tail (CT) of SARS SP contains a dibasic motif (KxHxx) that constitutes for an endoplasmic reticulum (ER) retrieval signal which retains the full-length SARS-SP in the lumen of the ER-Golgi intermediate compartment (ERGIC) (30, 38). Deletion of 17 aa at the carboxyl-terminal in the CT of SARS SP was able to increase SP transported to the surface of viral particles and the cells (5, 34). After rVSV-EboGPΔM-M2e/SPΔCa742 and rVSV-EboGPΔM-M2e/SPΔS2ΔC vectors were constructed, we then performed the rescue experiments in VeroE6 cells and successfully rescued rVSVΔG-EboGPΔM-M2e/DeltaSPΔCa742 and rVSVΔG-EboGPΔM-M2e/DeltaSPΔS2ΔC viruses (FIG. 2).

Then, we infected VeroE6 cells with rVSVΔG-EboGPΔM-M2e/DeltaSPΔCa742 and rVSVΔG-EboGPΔM-M2e/DeltaSPΔS2ΔC. After two days of infection, we collected the infected cells and non-infected cells. The presence of DeltaSPΔCa7, DeltaSPΔS2ΔC, and EboGPΔM-M2e were detected by indirect immunofluorescence assay with a human anti-SARS-CoV-2 SP (NTD) antibody or anti-influenza M2 antibody and the results revealed the presence of DeltaSPΔCa7, DeltaSPΔS2ΔC, and EboGPΔM-M2e in the infected cells, but not in non-infected cells (FIG. 2). Meanwhile, we checked the DeltaSPΔCa742, DeltaSPΔS2ΔC, and EboGPΔM-M2e expression with SDS-PAGE followed by WB with corresponding antibodies, as indicated in FIG. 2. Results showed that the expression DeltaSPΔCa742 (FIG. 2) and DeltaSPΔS2ΔC (FIG. 2) were detected. Also in the rVSVΔG-EboGPΔM-M2e/DeltaSPΔCa742 and rVSVΔG-EboGPΔM-M2e/DeltaSPΔS2ΔC infected VeroE6 cells, the EboGPΔM-M2e expression was detected (FIG. 2). Meanwhile, the VSV nucleocapsid (N) protein was detected in all rVSV infected cells (FIG. 2). All of these results demonstrated that abundant expression of DeltaSPΔCa742, DeltaSPΔS2ΔC, and EboGPΔM-M2e in the corresponding rVSV infected cells.

### Example 5—Generation of rVSV-Based Vaccines Expressing Both the Conserved M2 Ectodomain (M2e) of Influenza and SARS-CoV-2 Delta Spike Protein

Given that a new SARS-CoV-2 Delta variant (B11.617.2) had become a dominant variant that spread rapidly around the world [12], it was of interest to develop a vaccine that specifically targeted Delta and related variants. To this end, we generated several bivalent rVSV-based vaccines against both SARS-CoV-2 Delta variant and influenza virus. First, we generated a cDNA encoding SARS-CoV-2 Delta variant spike protein (SPDelta) containing a C-terminal 17 amino acid (aa) deletion (SPAC) (FIG. 1A, a). The deletion of 17 aa at the C-terminus of SP facilitates the transportation of SP to the plasma membrane and its assembly into virus because the assembly of SARS-CoV-2 occurs in the ER-Golgi intermediate compartment [34, 5]. To reduce the cytotoxic effect of SPΔC_{delta}[59] in the vaccine platform, we also introduced an isoleucine (1) to alanine (A) substitution at position 742 aa and named it SPΔC1 (FIG. 1A, a). The analyses revealed that the I742A point mutation in SPΔC1 significantly reduced pseudovirus infectivity (FIG. 1B) and syncytia formation compared to SPΔC_{Delta} in A549-ACE2 cells (FIGS. 1C and D). In SP_{Delta}ΔS2ΔC (named SPΔC2), a 381 aa fragment in the S2 domain (744-1124 aa) was further deleted (FIG. 1A, b). Meanwhile, we inserted a receptor-binding domain (RBD) from SARS-CoV-2 (wild type) SP into the Ebola glycoprotein (EboGPΔM) to replace the mucin-like domain (MLD) and named it EboGPΔM-RBD (ERBD) (FIG. 1A, c). Finally, we inserted cDNA encoding SPΔC1, SPΔC2 and ERBD into a recently reported rVSV-EM2e vaccine vector, which contains an EboGPΔM fused with four copies of influenza M2 ectodomain (24 aa) polypeptide (EboGPΔM-tM2e, or EM2) [53](FIG. 1E) and named them V-EM2e/SPΔC1, V-EM2e/SPΔC2, and V-EM2e/ERBD (FIG. 1E).

The established reverse genetics technology [76] was used to rescue the rVSV vaccine candidates, and three rVSV vaccine candidates, rVSV-EM2e/SPΔC1, rVSV-EMe2/SPΔC2 and rVSV-EM2e/ERBD, were successfully rescued, as described in Materials and Methods (FIG. 1C). To verify the replicating ability of the rescued rVSV vaccine candidates and their expressions of SPΔC1, SPΔC2, ERBD, and EM2e, respectively, we infected Vero E6 cell line with each rVSV and observed the cytopathic effect induced during the replication of each rVSV vaccine candidate (FIG. 2A) Also, the abundant expression of SPΔC1, SPΔC2, ERBD, and EM2e were detected in each corresponding rVSV-infected cells by immunofluorescence assay and the western blotting (WB) with corresponding antibody (FIGS. 2C and D). As expected, the expression of VSV nucleocapsid (N) protein was detected in all rVSV-infected cells (FIG. 2C, lower panel, Lanes 2 to 4).

### Example 6—Replication Attenuation and Different Cell Tropisms of Bivalent VSV Vaccine Candidates Compared to Wild-Type VSV

In our rVSV vaccine strategy, VSV-G was replaced by EM2e and SPAC or ERBD, which attenuated the pathogenicity of rVSV. Considering that rVSV is a replication-competent vector, it is necessary to investigate the replication ability and cell tropism of the above rVSV vaccine candidates. We therefore used a dose of 100 TCID50 to infect following cell lines: A549, a type II pulmonary epithelial cell line [60]; MRC-5, a human lung fibroblast cell line [74]; U251 MG, a glioblastoma cell line; CD4+ Jurkat T cells; human monocyte-derived macrophages (MDMs) and dendritic cells (DCs) (FIG. 3). We assessed the cytopathic effect (CPE) of rVSV vaccine candidates and assessed their growth kinetics. A comparison study revealed that 1) rVSV vaccine candidates were unable to infect CD4+ Jurkat T cells and MRC-5 cells, while wild-type VSV replicated efficiently in all tested cells and induced typical CPEs, such as cell rounding and detachment (FIG. 3A, B). 2) In A549 cells, U251 cells, MDMs and MDDCs, three rVSV vaccine candidates displayed positive infection but exhibited much slower replication kinetics and no significant CPE was observed compared to wild-type VSV during the testing period. This implies that these rVSV vaccine candidates have less replication ability and cytopathic effects. All of these data provided evidence supporting that the replication ability of these replicating rVSV vaccine candidates is highly attenuated compared to wild-type VSV.

### Example 7—Evaluation of Anti-SARS-CoV-2 and Anti-Influenza M2 Humoral Immune Responses Induced by Different Bivalent VSV Vaccine Candidates

To test whether the above bivalent VSV vaccine candidates could induce specific immune responses against SARS-CoV-2 and influenza M2 simultaneously, we administered two doses of vaccine (prime on Day 0 and boost on Day 14) in BALB/c mice with each bivalent VSV vaccine candidate intramuscularly (IM, 1×10⁸ TCID₅₀) or intranasally (IN, 1×10⁵ TCID₅₀). The potential adverse effects and the body weight of the mice were monitored daily for one week following vaccination. No changes were noticed clinically, and the steady weight gain of the vaccinated mice was comparable to that of the mock-immunized (PBS) group. Sera from immunized mice were collected on Days 14 and 28 post immunization, and the anti-SARS-CoV-2 RBD and anti-M2 antibody levels were measured using the corresponding antigen-coated ELISA as described in the Materials and Methods. The results showed that 1) IM immunization with V-EM2e/SPΔC1 or V-EM2e/SPΔC2 induced higher levels of circulating anti-SARS-CoV-2 RBD IgG and IgA antibodies than IN immunization (FIG. 4B-E); 2) V-EM2e/ERBD IM administration induced much lower levels of anti-SARS-CoV-2 RBD IgG antibodies than the other two rVSV vaccines (FIGS. 4B and C); 3) All vaccine candidates elicited high levels of anti-M2-specific IgG and IgA antibodies regardless of the route of administration (FIG. 4F-1); 4) IN immunization resulted in more complicated anti-RBD and anti-M2 antibody profiles. IN immunization with V-EM2/SPΔC1 induced slightly higher levels of anti-SARS-CoV-2 IgG and IgA antibodies than IN immunization with V-EM2/SPΔC2 (FIG. 4, B-E, compare bars 3 to 4). Conversely, the levels of anti-M2 IgG and IgA antibodies induced by V-EM2/SPΔC1 were slightly lower than those induced by rVSV-EM2/SPΔC2 (FIG. 4, compare F-I, compare bars 3 to 4). The underlying mechanism(s) is still unclear. Nevertheless, all of these observations indicate that IM- and IN-immunizations with V-EM2/SPΔC1 and V-EM2/SPΔC2 induced efficient anti-SARS-CoV-2 RBD and anti-M2 immune responses.

### Example 8—Vaccination with Bivalent VSV Vaccine Candidates Induced Potent Neutralizing Antibodies that Protect Against Infection with Various SARS-CoV-2 SP Pseudoviruses

The control of SARS-CoV-2 transmission relies on herd immunity among the human population, which can be obtained via infection-induced or vaccination-induced immunity. An ideal COVID-19 vaccine must be able to prevent SARS-CoV-2 infection by inducing a high level of neutralizing antibodies (nAbs). In this study, we tested whether the antibodies induced by different rVSV-based SP vaccine candidates could neutralize SARS-CoV-2 pseudoviruses (PVs) and prevent infection. Various single-round SpΔC-pseudoviruses expressing firefly luciferase (Luc) were produced by co-transfecting 293T cells with a HIV-Env⁻/Luc+ vector [75] and each of SPΔC-expressing plasmids, as described in M & M and indicated in FIG. 5. Neutralization assays showed that among the three IM immunization groups, the sera from mice immunized with V-EM2e/SPΔC1 contained the highest levels of neutralizing antibodies against SpΔC_{WT}- or SpΔC_{Delta}-PVs infections, while V-EM2e/ERBD immunization showed very low neutralizing activity (FIGS. 5A and B), which was consistent with the low level of anti-RBD IgG present in V-EM2e/ERBD immunization sera (FIGS. 4B and C). These results indicate that the full-length SP was the most efficient in stimulating neutralizing antibody production. As expected, the V-EM2e/SPΔC1- and V-EM2e/SPΔC2-immunized mice sera had higher neutralizing activity against SpΔC_{Delta}-PVs than SpΔC_{WT}-PVs (FIG. 5, compare B to A). Additionally, regardless of the route of administration (IM or IN), V-EM2e/SPΔC1 vaccination induced antibodies that were able to neutralize SpΔC_{B.1.617}-PV or SpΔC_{Beta′}-PV at levels similar with that of NAbs against SpΔC_{WT}-PV (FIGS. 5D and E). Given the new dominant VOC, Omicron variant, contains 32 mutations in SP and has a high immune escape ability [51, 52, 46], we therefore also assessed the neutralization activity of V-EM2e/SPΔC1-immunized mice sera against Omicron SpΔC_{Omic}-pseudoviruses. Results revealed that although the neutralizing activity of sera was significantly reduced as compared with NAbs against SpΔC_{Delta}-PV, the V-EM2e/SPΔC1 IM-immunized mice sera still retained over 10³ titer of NAb against SpΔC_{Omic}-PV infection (FIG. 5F), that was likely comparable with the NAb titers against 2019-nCoV in the sera of COVID-19 infected patients detected in a previously study [77]. We also noticed that the neutralizing antibody titers in sera from the IM-vaccinated groups were significantly higher than that in the IN-vaccinated groups. These results were expected because the immunization dose (1×10⁸ TCID₅₀/mouse) for the IM groups was 1000-fold higher than that (1×10⁵ TCID₅₀/mouse) for the IN groups. All tested immune sera did not show any neutralization activity against VSV-G-PV infection (FIG. 5C). In summary, V-EM2e/SPΔC1, which contained full-length Delta-SPAC, elicited high titers of neutralizing antibodies against Delta SP-pseudovirus infection and, to a less extent, against SPΔC_{WT}-, SpΔC_{Beta′}-, and SpΔC_{Omic}-pseudovirus infections in vitro.

### Example 9—Induction of Th1/2 Cytokines in Splenocytes from the Mice Immunized by the Bivalent VSV Vaccine Candidates

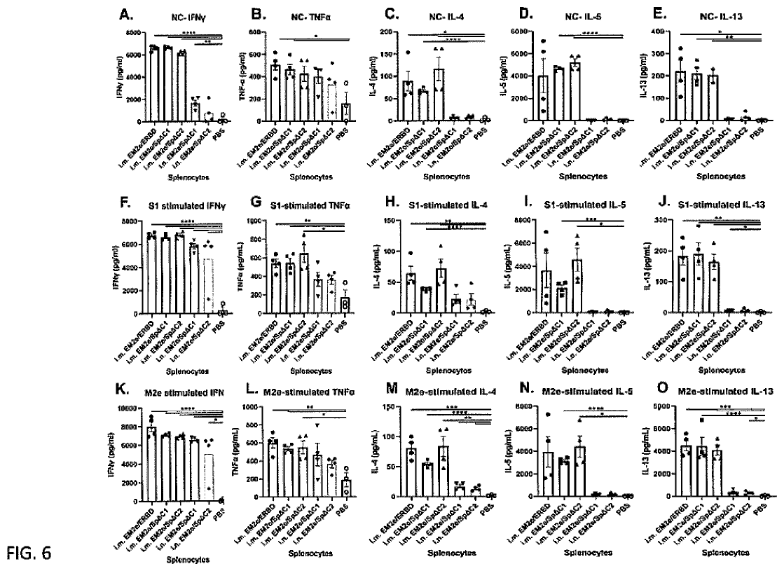

Effective vaccination involves induction of T helper cells that produce cytokines to shape subsequent humoral adaptive immune responses. We therefore collected splenocytes from the naïve animals and the immunized animals (via IM or IN injection route). They were cultured in the absence of any peptides (FIG. 6A-E), with the SARS-CoV-2 SP subunit 1 (S1) overlapping peptide pool (FIG. 6F-J) or with influenza M2e peptides (FIG. 6K-O). We examined the levels of specific cytokine production in the splenocyte culture medium to determine whether T cells were stimulated in the immunized mice.

As expected, we observed low/no level of Th1 cytokines (IFNγ and TNFα) and Th2 cytokines (IL-4, IL-5 and IL-13) in the splenocytes of PBS-treated control animals. In contrast, high level of Th cytokines were detected in the animals that were immunized with our vaccine candidates. For the IN-immunized mice splenocytes, slightly elevated Th1 cytokines (IFNγ and TNFα) were detected without peptide treatment, while the Th2 cytokines (IL-4, IL-5 and IL-13) in these groups were as low as those of the PBS group (FIG. 6A-E, compare bars 4-5 to bar 6). However, the stimulation with S1 or M2e peptides markedly elevated the secretion of IFNγ and, to a lesser extent, IL-4 from IN-immunized splenocytes compared with PBS control (FIG. 6, compare F and H, to A; K and M to C, bars 4 and 5), suggesting the S1/M2e-specific re-activation ability (memory) of these splenocytes. Further, the ratios of IFNγ (Th1) and IL-5 (Th2) of these IN-immunized mice after peptide-treatments were 25˜750 (FIG. S2D and G, bars 4-5), implying that IN immunization stimulated a very strong Th1-biased response.

For IM-immunized mice, both Th1 cytokines (IFNγ and TNFα) and Th2 cytokines (IL-4, IL-5 and IL-13) showed a statistically significant elevation in the splenocytes supernatants compared with those of the naïve PBS control group. Interestingly, such high levels of cytokine production were observed in the splenocytes cultures that had no peptide re-stimulation (FIG. 6A-E, compare bars 1-3 to bar 6). Addition of specific Ag (S1, M2e peptides) could not further augment the cytokine production in the splenocytes in vitro (FIG. 6A-E, bars 1-3 to F-O, bars 1-3). The ratios of Th1 cytokines and Th2 cytokines (such as IFNγ/IL-5 and TNFα/IL-4) of these IM-immunized mice were around 1˜15, indicating that the IM immunization stimulated a more Th1/Th2-balanced cellular response with a little Th1-bias. Collectively, above results suggested that our VSV-based vaccine candidates have good immunogenicity to elicit strong T-cell immune responses, and the administration via IM route can induce a Th1 bias, but relatively Th1/Th2-balanced immune response while the administration via IN route will induce a very strong Th1 biased response.

### Example 10—Immunization with V-EM2/SPΔC1 Protects Mice from Lethal H1N1 and H3N2 Influenza Virus Challenge

The above studies have demonstrated the strong humoral and cellular immune responses induced by V-EM2e/SPΔC1. We next investigated whether V-EM2e/SPΔC1 immunization could protect against influenza virus infection. Briefly, groups of 5 mice were vaccinated with V-EM2/SPΔC1 via either IM- or IN route and boosted on Day 14, while control mice received only PBS (IN) (FIG. 7A). For a mouse-adapted H3N2 virus challenge experiments, all mice were immunized with V-EM2/SPΔC1 via IN route, with (DD) or without (SD) boost on Day 14. On Day 28, we confirmed before challenge that high levels of anti-M2e antibodies were induced in all immunized mice in both vaccine delivery routes (FIG. 7B). Then, all mice were challenged with a fatal dose of the A/Puerto Rico/8/34 H1N1 strain (2.1×10³ TCID₅₀/mouse) or H3N2 virus (1.4×10⁴ TCID₅₀/mouse) intranasally as previously described [53]. Following challenge with either H1N1 strain or H3N2, a high morbidity rate was observed among the PBS group mice, exhibiting significant weight loss until death or reaching the end point for humane euthanasia (over 20% weight loss) within 5 or 6 days. (FIGS. 7C and F). In contrast, both the IM and IN groups of V-EM2/SPΔC1-vaccinated mice showed moderate weight loss (˜11%) until Day 4 and then almost full recovery by Days 8-10. The survival curve further indicated that both IM- and IN-immunized mice had 100% protection against H1N1 and H3N2 infections (FIGS. 7D and G). We also monitored the H1N1 or H3N2 viral loads in the lungs of two animals from each infected group. The result showed that the titer of H1N1 or H3N2 virus in PBS-treated mice reached approximately 3×10⁶ TCID50/gram or 5×10⁸ TCID50/gram of lung tissue at 5 or 6 days post-challenge. However, the virus titers in the immunized mice were only approximately 3×10³ TCID50/gram or 5×10⁴ TCID50/gram of lung tissue respectively, indicating that H1N1 and H3N2 virus replications were considerably suppressed in the lungs of immunized mice (FIGS. 7E and H). Interestingly, the results also indicate that a single-dose administration via IN achieved similar protection efficiency as that double-dose administration. Overall, these results provide strong evidence that both IM and IN vaccination with the bivalent vaccine V-EM2e/SPΔC1 were safe and effectively protected mice from lethal H1N1 and H3N2 influenza challenge.

### Example 11—V-EM2/SPΔC1 and V-EM2/SPΔC2 Protects Syrian Hamsters from SARS-CoV-2 Delta Virus Infection

Next, we investigated whether immunization with V-EM2/SPΔC1 and V-EM2/SPΔC2 could protect Syrian hamsters from SARS-CoV-2 virus infection. Briefly, three groups of 10 hamsters were vaccinated with either V-EM2/SPΔC1 or V-EM2/SPΔC2 via an IM route and boosted on Day 28, while control hamsters received only PBS (FIG. 8A). On Days 28 and 42 (prior to virus challenge), we monitored anti-SARS-CoV-2 SP IgG titers and results showed that one dose (1×10⁸ TCID50 per hamster) of either vaccine induced a strong anti-SP antibody response at 28 days post-vaccination (FIG. 8B). The V-EM2/SPΔC1 induced a two-fold higher median titer than V-EM2/SPΔC2 following the first dose, but this difference between the two vaccine groups was not significant. Interestingly, two weeks following the booster dose, V-EM2/SPΔC1 vaccinated animals only saw a small increase in antibody titer, while V-EM2/SPΔC2 immunized animals did not have an increase in median titer. Overall, these data indicate that a single dose of each rVSV vaccine is capable of inducing strong humoral responses against SARS-CoV-2 SP and that an additional dose provides a marginal boost for antibody responses, measured two weeks after the boost.

Fourteen days following the second vaccine dose all animals were challenged intranasally with the SARS-CoV-2 Delta variant. For two days following infection, animals in all groups showed slight weight loss before animals in both vaccine groups began to trend back toward their initial starting weights. Animals in the PBS group continued to lose weight until maximal weight loss seen on day 6, before recovering to the initial weight by day 12 (FIG. 8C). Significant differences in mean weight between groups, compared to PBS-administrated group, were detectable as early as day 2 post-infection in the V-EM2/SPΔC1 group and day 3 post-infection in the V-EM2/SPΔC2 group. Mean weight of the animals in the V-EM2/SPΔC1 group remained significantly higher than the PBS group through up to day 14. V-EM2/SPΔC2 animal weights remained significantly higher through day 9. Interestingly, V-EM2/SPΔC2 vaccinated animals, while being protected from the weight loss seen in control animals, did not see the overall weight gain throughout the course of infection seen in the V-EM2/SPΔC1 immunized animals, and their mean weights remained around their initial starting weight across the 14 days. Overall, the results clearly showed that each vaccine candidate was able to provide strong protection from weight loss observed during acute infection with Delta variant.

Following infection, oral swabs were collected to examine viral shedding in all animals on day three, and five animals from each group were euthanized on day five post-infection to examine the viral burden in respiratory tissues. On day three, both V-EM2/SPΔC1 and V-EM2/SPΔC2 vaccinated groups had significantly reduced levels of viral RNA in the oral swabs collected (FIG. 8D) indicating that vaccination may be capable of reducing viral shedding. On day five, infectious viral titers were examined in the nasal turbinates, upper lung, and lower lung of five animals from each group. While the majority of animals did not have infectious SARS-CoV-2 in the nasal turbinates, both groups had significantly reduced viral titers in the upper lung, and V-EM2/SPΔC1 animals had reduced levels in the lower lung as well (FIG. 8E). These experiments provide strong evidence that the vaccination with either V-EM2/SPΔC1 or V-EM2/SPΔC2 is capable of providing protection in the upper and lower airways, with significantly reduced viral replication, leading to reduced disease and viral shedding.

### Example 12—Materials and Methods

### Plasmid Constructions.

The gene encoding SPΔC_{Delta} was amplified from the previously described plasmid pCAGGS-SPΔC_{Delta} [59] and the I742A mutation was introduced by site-directed mutagenesis technique with, 5′-primers 5_TGTACAATGTATGCATGCGGAGACAGC (SEQ ID NO:13), and 3′-primer, 5_GCTGTCTCCGCATGCATACATTGTACA (SEQ ID NO: 14). Then, the amplified SPΔC_{Delta}-I742A gene was cloned at XhoI and NheI sites of an rVSV-based influenza vaccine vector, rVSV-EAM-M2e [53], and the constructed plasmid was named rVSV-EM2e/SPΔC1. To construct rVSV-EM2e/SPΔC2, we used a two-step PCR technique to generate cDNA that carried an additional 381 aa deletion in the S2 region of SPΔC_{Delta} (FIG. 1A, b), and the amplified SPΔC2-encoding cDNA was also cloned into the rVSV-EAM-M2e vector using the same restriction enzymes, yielding rVSV-EM2e/SPΔC2. To construct rVSV-EM2e/ERBD, a cDNA fragment encoding the receptor binding domain (RBD) of SARS-CoV-2 (Wuhan-Hu-1, GenBank accession No. MN908947) spike protein was amplified from a pCAGGS-nCoVSP plasmid [5] and inserted in pCAGGS-EboGPΔM at the MLD region [53]. Then, the EboGPΔM-RBD cDNA (FIG. 1A, d) was cloned into the XhoI and NheI sites of the rVSV-EAM-EM2e vector and named rVSV-EM2e/ERBD (FIG. 1B). To construct pCAGGS-SPΔCBeta′ and pCAGGS-SPΔC_{B.1.617}, the mutations K417N, E484K, N501Y and D614G (for SPΔCBeta′) and L452R, E484Q and D614G (for SPΔC_{B.1.617}) were introduced into the pCAGGS-nCoVSPΔC plasmid [59] by site-directed mutagenesis. For constructing pCAGGS-SPΔCo_{mic} expressing plasmid, the gene encoding SPΔC_{Omic}, as described previously [51], was synthesized (Genescript) and cloned into the pCAGGS plasmid. All the inserted SPAC transgenes in rVSV vectors and various pCAGGS-SPAC plasmids were confirmed by sequencing.

### Cells, Antibodies, Chemicals and Viruses.

A human embryonic kidney cell line (HEK293T), a human lung type 11 pulmonary epithelial cell line (A549), a human lung fibroblast cell line (MRC-5), a human glioblastoma-derived cell line (U251 GM), VeroE6 and MDCK cell line were cultured in Dulbecco's modified Eagle's medium, minimum essential medium (MEM) or DMEM/F-12 medium (21331-020, Gibco). CD4⁺ Jurkat cells were cultured in RPMI-1640 medium. A549 cells expressing the ACE2 receptor (A549_{ACE2}) were described previously [59]. Human monocyte-derived macrophage MDMs and dendritic cells (MDDCs) were prepared from human peripheral blood mononuclear cells (hPBMCs) isolated from healthy donors following procedures as described previously [65]. All cell lines were grown in cell culture medium supplemented with 10% fetal bovine serum (FBS), 1× L-glutamine and 1% penicillin and streptomycin. The antibodies used in the study included the rabbit polyclonal antibody against SARS-CoV-2 SP/RBD (Cat #40150-R007, Sino Biological), anti-SARS-CoV-2 S-NTD antibody (E-AB-V1030, Elabscience), anti-M2 monoclonal antibody (14C2: sc-32238, Santa Cruz Biotech), and anti-VSV-Nucleoprotein, clone 10G4 (Cat #MBAF2348, EMD Millipore Corp). The HIV-1 p24 ELISA Kit was obtained from the AIDS Vaccine Program of the Frederick Cancer Research and Development Center. Recombinant SARS-CoV-2 proteins or peptides used in this paper include S1-RBD peptides (RayBiotech, Cat #230-30162) and S1 overlapping peptide pool (JPT Peptide Technologies, Germany, Cat #PM-WCPV—S-SU1-1; 166 peptides; 15mers with 11 aa overlap). Influenza M2e peptide and mouse-adapted strain A/Puerto Rico/8/34 (H1N1) were described previously [53].

rVSV Rescue and Virus Growth Kinetics Experiments.

Replication-competent rVSV was recovered in 293T-Vero E6 co-cultured cells as described previously [53]. All three bivalent VSV vaccine candidates were propagated and titrated on Vero E6 cells. To examine the growth kinetics of bivalent rVSV, cell lines were grown to confluency in a 24-well plate and infected in duplicate with VSVwt, V-EM2e/SPΔC1, rV-EM2e/SPΔC2 or V-EM2e/ERBD at a dose of 100 TCID50. After 2 hrs of incubation, the cells were washed and cultured in DMEM or RPMI containing 2% FBS. The supernatants were collected at 24, 48, 72 and 96 hours. The titers of rVSV in the supernatant were determined by the TCID50 method on Vero E6 cells in 96-well plates.

To detect the expression of EM2, Delta SPΔC, RBD, and other viral proteins in cells, rVSV-infected cells were lysed and analyzed by SDS-PAGE and WB with anti-M2e (14C2), anti-SARS-CoV-2-RBD, or anti-VSV N antibodies.

### Immunofluorescence Assay and Syncytia Formation Assay

As previously described [53], Vero E6 cells were grown on glass coverslips (12 mm²) in 24-well plates and infected with V-EM2e/SPΔC1, V-EM2e/SPΔC2 or V-EM2e/ERBD for 48 hours. After infection, cells on the coverslip were fixed with 4% paraformaldehyde for 15 minutes and permeabilized with 0.2% Triton X-100 in PBS. The glass coverslips were then incubated with primary antibodies specific for M2e or SP/RBD followed by corresponding FITC-conjugated secondary antibodies. Cells were viewed under a computerized Axiovert 200 inverted fluorescence microscope (Zeiss).

To test SARS-CoV-2 SPDelta-mediated syncytia formation, 293T cells were transfected with various SPAC plasmids using Lipofectamine 2000. After 24 hrs, the cells were washed, resuspended and mixed with A549ACE2 cells at a 1:3 ratio and plated into 12-well plates. At different time points, syncytium formation was observed, counted and imaged by bright-field microscopy with an Axiovert 200 fluorescence microscope.

### Virus Production and Infection Experiments.

SARS-CoV-2 SPΔC-PVs (SPΔCwt-, SPΔC_{Delta}-, SPΔC_{Delta}-a742-PVs) were produced by co-transfecting 293T cells with each of the pCAGGS-SPAC plasmids, pCMVΔ8.2 and Gluc expressing HIV vector ΔRI/E/Gluc [5]. After 48 hrs of transfection, cell culture supernatants were collected, VPs were purified and quantified by HIV-1 p24 amounts using an HIV-1 p24 ELISA, as described previously [59]. To measure the infection of SPΔC-pseudotyped VPs, equal amounts of each SPΔC-PV (as adjusted by p24 levels) were used to infect A549_{ACE2}, the supernatants were collected, and the viral infection levels were monitored by measuring Gaussia luciferase (Gluc) activity.

### Cell-Based Pseudovirus Neutralization Assays

Different SARS-CoV-2 SP pseudoviruses expressing luciferase were prepared and titrated as follows: HIV-based SARS-CoV-2 SP pseudoviruses (PVs) expressing luciferase (Luc) were produced by co-transfection of 293T cells with an HIV vector (pNL4-3-R-E-Luc) [62] and each pCAGGS-SPAC or pCAGGS-VSV-G plasmid by using polyetherimide (PEI) transfection in a 6-well plate. The supernatants were harvested at 72 h post-transfection, passed through a 0.45 μm filter, aliquoted and titrated on A549 cells expressing human ACE2 (A549/hACE2).

The pseudovirus neutralization assay was performed on A549/hACE2 cells according to previously reported methods with some modifications [78, 79]. Briefly, inactivated mouse sera of the same experimental group were pooled together. SPAC pseudotyped Luc-PVs (PV-Luc-SpΔC) and control VSV-G-pseudotyped Luc-PV-Luc (25 μL, ˜104 RLU) were pre-incubated with 2× serially diluted mouse sera (25 μL) in a 96-well plate for 1.5 h at room temperature with gentle shaking. Then, A549/hACE2 (1.25×10⁴ cells/well, 50 μL) and polybrene (final conc. 5 μg/mL) were plated in the above wells containing a mix of pseudovirus and sera. After gentle shaking for 10 min, the cells were incubated at 37° C. overnight. The supernatant was removed the next day, and fresh medium was added. At 48˜66 hrs post-infection, cells were washed and lysed, and the luciferase RLU in lysate was measured. The RLU percentages (%) were obtained by comparing the RLU of tested samples to the RLU of the control samples after subtraction of the background RLUs (cell only/virus only). The neutralizing titers or half-maximal inhibitory dilution (ID50) were defined as the reciprocal of the serum maximum dilution that reduced RLU by 50% relative to no-serum (virus and cell) controls. The ID50 was calculated by using sigmoid 4PL interpolation with GraphPad Prism 9.0. All data were from at least three experiments and are shown as the means±standard error of the means (SEMs).

### Mouse Immunization and Viral Challenge

Female BALB/c mice aged 6-8 weeks used in this study were obtained from the Central Animal Care Facility, University of Manitoba (with animal study protocol approval No. 20-034). For rVSV immunization, mice (five per group) were immunized intramuscularly (IM, 1×10⁸ TCID₅₀) or intranasally (IN, 1×10⁵ TCID₅₀) with rVSV vaccine candidates on Day 0 and boosted on Day 14. Mice were sacrificed on Day 28, and spleens were harvested. Blood samples were collected on Days 13 and 28. For influenza virus challenge in mice, the mouse-adapted strain A/Puerto Rico/8/34 (H1N1) was used. Three groups of mice (5 for each group) were IM-immunized with 1×10⁸ TCID₅₀ or IN-immunized with 1×10⁵ TCID₅₀ of V-EM2e/SPΔC1 or PBS on Day 0 and boosted on Day 14. On Day 28, all the mice were intranasally infected with H1N1 virus (2.1×10¹ PFU/mouse) or with H3N2 virus (1.4×10⁴ PFU). Weight and survival of the mice were monitored daily for 2 weeks after the challenge. Additionally, 5 to 6 days post-challenge, the mice from the PBS group and two mice from the vaccination group were sacrificed, and the lungs were collected and immediately stored at −80° C. The lung was homogenized using a tissue grinder and centrifuged at 5,000 rpm. The supernatant was used for titration in MDCK cells according to the method described previously [80, 81].

The SARS-CoV-2 challenge experiments were carried out at the National Microbiology Laboratory (NML) of the Public Health Agency of Canada and approved by the Animal Care Committee at the Canadian Science Center for Human and Animal Health. All infectious work was performed under biosafety level 3 (BSL-3) conditions or higher. Different groups of ten Syrian Golden hamsters (five male and five female) were anaesthetized and administered with 10⁸ PFU of either V-EM2e/SPΔC1 or V-EM2e/SPΔC2, or PBS via intramuscular injection. Then 28 days later, animals were given their second immunization. Animals were recovered and monitored daily for any adverse signs following vaccine administration. After 14 days following their second dose, hamsters were moved into BSL-4 and then anaesthetized and intranasally infected with 8.9×10⁴ TCID₅₀/100 μL of the SARS-CoV-2 delta variant (SARS-CoV-2; B.1.617.2; hCoV-19/Canada/ON-NML-63169/2021 GISAID accession EPI_ISL_8439375). After infection, animals were weighed and monitored daily throughout the course of infection (14 days). On day three post-infection, oral swabs were performed on all animals. Groups of five animals (3 male and 2 female) from each experimental group were euthanized on day five post-infection for examination of viral burden in the tissues.

### Enzyme-Linked Immunosorbent Assay (ELISA) for Measurement of Anti-SARS-CoV-2-SP/RBD or Anti-Influenza M2e Antibody Levels in Immunized Mouse Sera

Anti-SARS-CoV-2-SP/RBD antibodies and anti-influenza M2 antibodies in mouse sera were determined by ELISA, as previously described with some modifications [53]. Briefly, ELISA plates (NUNC Maxisorp, Thermo Scientific) were coated with 100 μl of recombinant RBD protein or M2e peptide (0.75 μg/ml or 0.5 μg/ml, respectively) in coupling buffer (0.05 M sodium carbonate-bicarbonate, pH 9.6) overnight at 4° C. Then, the different dilutions of serum samples were incubated in the coated plate for 2 hrs at 37° C. followed by the addition of horseradish peroxidase-conjugated goat anti-mouse IgG or IgA for 1 h at 37° C. Finally, TMB (Mandel Scientific) was added, and the absorbance at 450 nm (OD450) was measured [82]. To determine the endpoint titers, 100 μl of 3× serially diluted sera was used to measure the OD450. The endpoint titer is designated as the reciprocal of the highest dilution of a serum that has an OD450 above the cutoff (10× negative control) and is calculated by using sigmoid 4PL interpolation with GraphPad Prism 9.0. For detection of anti-SARS-CoV-2 humoral responses, 96-well enzyme-linked immunosorbent assay (ELISA) low binding plates were coated overnight with 100 ng of purified SARS-CoV-2 spike. Then, hamster sera were diluted 1:100, then serially diluted two-fold and added into the ELISA plates for 1 hour incubation at 37° C. After extensive washing plates were further incubated with goat-anti hamster IgG secondary antibody for 1 hour at 37° C. Plates were then washed and added 100 μl of TMB substrate (Life Technologies) for 15 minutes incubation. Then, the plate reaction was stopped by adding 100 μl of 1 M H2SO4 solution and absorbance was analyzed on a Synergy (BioTek) microplate reader at 450 nm wavelength.

### Vaccine Candidates—Induced T Cell Responses

The mice were vaccinated according to the schedule described in FIG. 4A and sacrificed on Day 28 (2 weeks after booster). To assess general T cell reactivity, mouse splenocytes were collected as described previously [4] and plated in 48-well plates (2×10⁶/200 μl per well) in RPMI (no-peptide control) or incubated with a SARS-CoV-2 S1 overlapping peptide pool or with the influenza virus M2e peptide (1 μg/ml for each peptide). The PMA/ionomycin cocktail (Invitrogen, 81 pM/1.34 μM) served as a positive control. To measure the extracellular cytokines released from splenocytes, the supernatants were collected after 4 days and stored at −70° C. The Meso Scale Discovery (MSD) immunoassay was performed on a customized mouse U-plex Biomarker Group1 Assays kit (Mesoscale Discovery, USA) to determine the cytokines (IFN-γ, TNF-α, IL-4, IL-5 and IL-13) and analyzed on the MESO Quickplex SQ120 instrument following the manufacturer's instructions.

### Measurement of Viral Burden in the Tissues

For viral RNA detection in oral swabs viral RNA was extracted with the QIAamp Viral RNA Mini kit (Qiagen) according to manufacturer's instructions. Detection of SARS-CoV-2 E gene was performed using TaqPath 1-Step Multiplex Master Mix kit (Applied Biosystems) and was carried out on a QuantStudio 5 real-time PCR system (Applied Biosystems), as per manufacturer's instructions. RNA was reverse transcribed and amplified using the primers reported by the WHO and include E_Sarbeco_F1 (5′-ACAGGTACGTTAATAGTTAATAGCGT-3′) (SEQ ID NO: 15) and E_Sarbeco_R2 (5′-ATATTGCAGCAGTACGCACACA-3′) (SEQ ID NO:16) and probe E_Sarbeco_P1 (5′-FAM-ACACTAGCCATCCTTACTGCGCTTCG-BBQ-3′) (SEQ ID NO: 17). A standard curve for each plate using synthesized DNA was used for the quantification of viral genome copy numbers per mL of media.

For detection of infectious SARS-CoV-2 Delta virus, influenza H1N1 and H3N2 viruses in tissues, thawed tissue samples were weighed and placed in 1 ml of MEM supplemented with 1% heat-inactivated bovine growth serum, 1× L-glutamine, and 1× penicillin-streptomycin, along with a 5 mm stainless steel bead. Tissues were homogenized in a Bead Ruptor Elite Bead Mill Homogenizer (Omni International) at 4 m/s for 30 seconds then clarified by centrifugation at 1500×g for 10 minutes. Samples were serially diluted 10-fold in the same media as above and dilutions were then added to 96-well plates of 95% confluent Vero cells containing 50 mL of the same medium in replicates of three and incubated for 5 days at 37° C. with 5% CO2. Plates were scored for the presence of cytopathic effect on day 5 after infection. TCID50 titers were calculated using the Reed and Meunch Method [83].

### Statistics

Statistical analysis of antibody/cytokine levels was performed using the unpaired t test (considered significant at P≥0.05) by GraphPad Prism 5.01 software. The statistical analysis of neutralizing antibodies was performed using the one-way ANOVA multiple comparison test followed by Tukey's test by GraphPad Prism.

While the preferred embodiments of the invention have been described above, it will be recognized and understood that various modifications may be made therein, and the appended claims are intended to cover all such modifications which may fall within the spirit and scope of the invention.

### REFERENCES

1. Abdollahi, E., D. Champredon, J. M. Langley, A. P. Galvani, and S. M. Moghadas. 2020. Temporal estimates of case-fatality rate for COVID-19 outbreaks in Canada and the United States. CMAJ: Canadian Medical Association journal=journal de l'Association medicale canadienne 192:E666-E670.
2. Abiola, O., Z. J. Ao, M. Mahmoudi, G. Kobinger, and Y. X j. 2009. Dendritic cells/macrophages targeting feature of Ebola glycoprotein and its potential as immunological facilitator for antiviral vaccine approach. Microorganisms 7:402-425.
3. Ahmed, M. U., M. Hanif, M. J. Ali, M. A. Haider, D. Kherani, G. M. Memon, A. H. Karim, and A. Sattar. 2020. Neurological Manifestations of COVID-19 (SARS-CoV-2): A Review. Frontiers in neurology 11:518.
4. Ao, Z., L. Wang, E. J. Mendoza, K. Cheng, W. Zhu, E. A. Cohen, K. Fowke, X. Qiu, G. Kobinger, and X. Yao. 2019. Incorporation of Ebola glycoprotein into HIV particles facilitates dendritic cell and macrophage targeting and enhances HIV-specific immune responses. PloS one 14:e0216949.
5. Ao, Z. j., M. Chan, M. Ouyang, T. Abiola, M. Mahmoudi, D. Kobasa, and X. J. Yao. 2021. Identification and evaluation of the inhibitory effect of Prunella vulgaris extract on SARS-coronavirus 2 virus entry. PloS one:https://doi.org/10.1371/journal.pone.0251649
6. Ao, Z. j., T. Olukitibi, L. j. Wang, H. Azizi, M. Mahmoudi, G. Kobinger, and X. j. Yao 2021. Development and Evaluation of an Ebola Virus Glycoprotein Mucin-Like Domain Replacement System as a New Dendritic Cell-Targeting Vaccine Approach against HIV-1. Journal of virology 95: July 12; 95(15)::e0236820. PMID: 34011553.
7. Baud, D., X. Qi, K. Nielsen-Saines, D. Musso, L. Pomar, and G. Favre. 2020. Real estimates of mortality following COVID-19 infection. The Lancet. Infectious diseases 20:773.
8. Blanton, L., V. G. Dugan, A. I. A. Elal, N. Alabi, J. Barnes, L. Brammer, A. P. Budd, E. Burns, C. N. Cummings, and S. Garg. 2019. Update: Influenza Activityâ€″ United States, Sep. 30, 2018â€″ Feb. 2, 2019. Morbidity and Mortality Weekly Report 68:125.
9. Blut, A. 2009. Influenza virus. Transfusion Medicine and Hemotherapy 36:32.
10. Cummings, J. F., M. L. Guerrero, J. E. Moon, P. Waterman, R. K. Nielsen, S. Jefferson, F. L. Gross, K. Hancock, J. M. Katz, and V. Yusibov. 2014. Safety and immunogenicity of a plant-produced recombinant monomer hemagglutinin-based influenza vaccine derived from influenza A (H1N1) pdm09 virus: a Phase 1 dose-escalation study in healthy adults. Vaccine 32:2251-2259.
11. DeBuysscher, B. L., D. Scott, A. Marzi, J. Prescott, and H. Feldmann. 2014. Single-dose live-attenuated Nipah virus vaccines confer complete protection by eliciting anti-bodies directed against surface glycoproteins. Vaccine 32.
12. Dougherty, K., M. Mannell, O. Naqvi, D. Matson, and J. Stone. 2021. SARS-CoV-2 B.1.617.2 (Delta) Variant COVID-19 Outbreak Associated with a Gymnastics Facility—Oklahoma, April-May 2021. Morbidity and Mortality Weekly Report 70 1104.
13. Emanuel J, Callison J, Dowd K A, F. Pierson T C, eldmann H, and M. A. 2018. A VSV-based Zika virus vaccine protects mice from lethal challenge. Sci Rep. 8:11043.
14. Fan, J., X. Liang, M. S. Horton, H. C. Perry, M. P. Citron, G. J. Heidecker, T. M. Fu, J. Joyce, C. T. Przysiecki, P. M. Keller, V. M. Garsky, R. Ionescu, Y. Rippeon, L. Shi, M. A. Chastain, J. H. Condra, M. E. Davies, J. Liao, E. A. Emini, and J. W. Shiver. 2004. Preclinical study of influenza virus A M2 peptide conjugate vaccines in mice, ferrets, and rhesus monkeys. Vaccine 22:2993-3003.
15. Feldmann, H., F. Feldmann, and A. Marzi. 2018. Ebola: lessons on vaccine development. Annu. Rev. Microbiol. 72:423-446.
16. Grohskopf, L. A., and F. M. Munoz. 2018. 2018-2019 Recommendations for influenza prevention and treatment in children: an update for pediatric providers.
17. Jaimes, J. A., J. K. Millet, and G. R. Whittaker. 2020. Proteolytic Cleavage of the SARS-CoV-2 Spike Protein and the Role of the Novel S1/S2 Site. iScience 23:101212.
18. Kedzierska, K., C. Van de Sandt, and K. R. Short. 2018. Back to the future: lessons learned from the 1918 influenza pandemic. Frontiers in Cellular and Infection Microbiology 8:343.
19. Kim, G. U., M. J. Kim, S. H. Ra, J. Lee, S. Bae, J. Jung, and S. H. Kim. 2020. Clinical characteristics of asymptomatic and symptomatic patients with mild COVID-19. Clinical microbiology and infection: the official publication of the European Society of Clinical Microbiology and Infectious Diseases 26:948 e941-948 e943.
20. Kim, H. J., F. M. F., and D. J. Clemens. 2021. Looking beyond COVID-19 vaccine phase 3 trials. Nature medicine 27:205-211.
21. Kim, J. H., Marks F., and C. J. D. 2021. Looking beyond COVID-19 vaccine phase 3 trials. Nature Medicine 27:205-211.
22. Korber B., F. W., Gnanakaran S., Yoon H., Theiler J., Abfalterer W., Foley B., Giorgi E E., Bhattacharya T., Parker M D., Partridge D G., Evans C M., Freeman T M., de Silva T I, LaBranche C. C., and Montefiori D C. 2020. Spike mutation pipeline reveals the emergence of a more transmissible form of SARS-CoV-2. bioRxiv https://doi.org/10.1101/2020.04.29.069054.
23. Li, B. S., Deng A P, K. L., K b., Y. Hu, and e. al. 2021. Viral infection and transmission in a large, well-traced outbreak caused by the SARS-CoV-2 Delta variant. MedRxiv July 23: https://doi.org/10.1101/2021.1107.1107.21260122
24. Li, Q., X. Guan, P. Wu, X. Wang, L. Zhou, Y. Tong, R. Ren, K. S. M. Leung, E. H. Y. Lau, J. Y. Wong, X. Xing, N. Xiang, Y. Wu, C. Li, Q. Chen, D. Li, T. Liu, J. Zhao, M. Liu, W. Tu, C. Chen, L. Jin, R. Yang, Q. Wang, S. Zhou, R. Wang, H. Liu, Y. Luo, Y. Liu, G. Shao, H. Li, Z. Tao, Y. Yang, Z. Deng, B. Liu, Z. Ma, Y. Zhang, G. Shi, T. T. Y. Lam, J. T. Wu, G. F. Gao, B. J. Cowling, B. Yang, G. M. Leung, and Z. Feng. 2020. Early Transmission Dynamics in Wuhan, China, of Novel Coronavirus-Infected Pneumonia. The New England journal of medicine 382:1199-1207.
25. Lin, L., X. Jiang, Z. Zhang, S. Huang, Z. Zhang, Z. Fang, Z. Gu, L. Gao, H. Shi, L. Mai, Y. Liu, X. Lin, R. Lai, Z. Yan, X. Li, and H. Shan. 2020. Gastrointestinal symptoms of 95 cases with SARS-CoV-2 infection. Gut 69:997-1001.
26. Liu, W., P. Zou, J. Ding, Y. Lu, and Y. H. Chen. 2005. Sequence comparison between the extracellular domain of M2 protein human and avian influenza A virus provides new information for bivalent influenza vaccine design. Microbes and infection 7:171-177.
27. Lizhou Zhang, C. B. J., Huihui Mou, Amrita Ojha, Erumbi S Rangarajan, Tina Izard, Michael Farzan, Hyeryun Choe. 2020. The D614G mutation in the SARS-CoV-2 spike protein reduces S1 shedding and increases infectivity bioRxiv 06.12:148726.
28. Lu, R., X. Zhao, J. Li, P. Niu, B. Yang, H. Wu, W. Wang, H. Song, B. Huang, N. Zhu, Y. Bi, X. Ma, F. Zhan, L. Wang, T. Hu, H. Zhou, Z. Hu, W. Zhou, L. Zhao, J. Chen, Y. Meng, J. Wang, Y. Lin, J. Yuan, Z. Xie, J. Ma, W. J. Liu, D. Wang, W. Xu, E. C. Holmes, G. F. Gao, G. Wu, W. Chen, W. Shi, and W. Tan. 2020. Genomic characterisation and epidemiology of 2019 novel coronavirus: implications for virus origins and receptor binding. Lancet January 30 S0140-6736(0120)30251-30258.
29. Marzi, A., Robertson S J, Haddock E, Feldmann F, Hanley P. W, Scott D. P, Strong J. E, Kobinger G, Best S. M, and H. F. H. 2015. VSV-EBOV rapidly protects macaques against infection with the 2014/15 Ebola virus outbreak strain. Science 349:739-742.
30. McBride, C. E., J. Li, and C. E. Machamer. 2007. The cytoplasmic tail of the severe acute respiratory syndrome coronavirus spike protein contains a novel endoplasmic reticulum retrieval signal that binds COPI and promotes interaction with membrane protein. Journal of virology 81:2418-2428.
31. Nickol, M. E., and J. Kindrachuk. 2019. A year of terror and a century of reflection: perspectives on the great influenza pandemic of 1918â€″ 1919. BMC infectious diseases 19:117.
32. Organization, W. H. 2020. Estimating mortality from COVID-19 Scientific brief, 4 Aug. 2020. https://http://www.who.int/publications/i/item/WHO-2019-nCoV-Sci-Brief-Mortality-2020.1.
33. Pavlovic, J., T. Zurcher, O. Haller, and P. Staeheli. 1990. Resistance to influenza virus and vesicular stomatitis virus conferred by expression of human MxA protein. J. Virol.:3370-3375.
34. Petit, C. M., J. M. Melancon, V. N. Chouljenko, R. Colgrove, M. Farzan, D. M. Knipe, and K. G. Kousoulas. 2005. Genetic analysis of the SARS-coronavirus spike glycoprotein functional domains involved in cell-surface expression and cell-to-cell fusion. Virology 341:215-230.
35. Polack, P. F., J. S. Thomas, N. Kitchin, J. Absalon, et al., and f. t. C. C. T. Group. 2020. Safety and Efficacy of the BNT162b2 mRNA Covid-19 Vaccine List of authors. The New England journal of medicine 383:2603-2615.
36. Raj, V. S., A. D. M. E. Osterhaus, R. A. M. Fouchier, and B. L. Haagmans. 2014. MERS: emergence of a novel human coronavirus. Current opinion in virology 5:58-62.
37. Rose N F, Marx P. A, Luckay A, Nixon D. F, W. Moretto, Donahoe S M, Montefiori D, Roberts A, Buonocore L, and Rose J K. 2001. An effective AIDS vaccine based on live attenuated vesicular stomatitis virus recombinants. Cell 106:539-549.
38. Sadasivan, J., M. Singh, and J. D. Sarma. 2017. Cytoplasmic tail of coronavirus spike protein has intracellular targeting signals. Journal of biosciences 42:231-244.
39. Saelens X. 2019. The Role of Matrix Protein 2 Ectodomain in the Development of Universal Influenza Vaccines. The Journal of Infectious Diseases. 219:S68-S74.
40. Su, S., G. Wong, W. Shi, J. Liu, A. C. K. Lai, J. Zhou, W. Liu, Y. Bi, and G. F. Gao. 2016. Epidemiology, genetic recombination, and pathogenesis of coronaviruses. Trends in microbiology 24:490-502.
41. Suder, E., W. Furuyama, H. Feldmann, A. Marzi, and E. de Wit. 2018. The vesicular sto-matitis virus-based Ebola virus vaccine: from concept to clinical trials. Hum. Vaccin. Immunother. 14:2107-2113.
42. Uranowska, K., J. Tyborowska, A. Jurek, B. a. Szewczyk, and B. Gromadzka. 2014. Hemagglutinin stalk domain from H5N1 strain as a potentially universal antigen. Acta Biochimica Polonica 61.
43. Valkenburg, S. A., V. V. A. Mallajosyula, O. T. W. Li, A. W. H. Chin, G. Carnell, N. Temperton, R. Varadarajan, and L. L. M. Poon. 2016. Stalking influenza by vaccination with pre-fusion headless HA mini-stem. Scientific reports 6:22666.
44. Wang, L., A. Hess, T. Z. Chang, Y. C. Wang, J. A. Champion, R. W. Compans, and B. Z. Wang. 2014. Nanoclusters self-assembled from conformation-stabilized influenza M2e as broadly cross-protective influenza vaccines. Nanomedicine: Nanotechnology, Biology and Medicine 10:473-482.
45. Weir, J. P., and M. F. Gruber. 2019. An overview of the regulation of influenza vaccines in the United States. Influenza and other respiratory viruses 10:354-360.
46. Witko, S. E., C. S. Kotash, R. M. Nowak, J. E. Johnson, L. A. Boutilier, K. J. Melville, S. G. Heron, D. K. Clarke, A. S. Abramovitz, R. M. Hendry, M. S. Sidhu, S. A. Udem, and C. L. Parks. 2006. An efficient helper-virus-free method for rescue of recombinant paramyxoviruses and rhadoviruses from a cell line suitable for vaccine development. Journal of virological methods 135:91-101.
47. Yao, X. J., Z. J. Ao, and O. Abiola. 2019. Dendritic Cell-Targeting Universal Vaccine for Influenza Infection. Patent application USPTO Ref No. U.S. Ser. No. 62/923,842.
48. Yao, X. j. A., Z. Kobinger, G. 2019. Ebola virus and Marburg virus glycoprotein mucin-like domain replacement expression system used as new vaccine approaches (PCT/CA2018/051577). https://patentscope.wipo.int/search/en/detail.jsf?docld=WO2019113688&_cid=P12-K5Y7L0-43129-1 WO/2019/113688:20.06.2019.
49. Zhang, Y. Z. 2020. Severe acute respiratory syndrome coronavirus 2 isolate Wuhan-Hu-1, complete genome. GenBank: MN908947.3.
50. Zhou, Z., H. Kang, S. Li, and X. Zhao. 2020. Understanding the neurotropic characteristics of SARS-CoV-2: from neurological manifestations of COVID-19 to potential neurotropic mechanisms. Journal of neurology 267:2179-2184.
51. Ma W, Yang J, Fu H, Su C, Yu C, Wang Q, et al. Genomic perspectives on the emerging SARS-CoV-2 omicron variant. Genomics Proteomics Bioinformatics. 2022. Epub 2022/01/17. doi: 10.1016/j.gpb.2022.01.001. PubMed PMID: 35033679; PubMed Central PMCID: PMCPMC8791331.
52. Dejnirattisai W, Huo J, Zhou D, Zahradnik J, Supasa P, Liu C, et al. SARS-CoV-2 Omicron-B.1.1.529 leads to widespread escape from neutralizing antibody responses. Cell. 2022; 185(3):467-84.e15. doi: https://doi.org/10.1016/j.cell.2021.12.046.
53. Emanuel J, Callison J, Dowd K A, Pierson T C F, eldmann H, A. M. A VSV-based Zika virus vaccine protects mice from lethal challenge. Sci Rep 2018; 8(1):11043.
54. Case J B, Rothlauf P W, Chen R E, Kafai N M ea. Replication-Competent Vesicular Stomatitis Virus Vaccine Vector Protects against SARS-CoV-2-Mediated Pathogenesis in Mice. Cell host & microbe. 2020; 28(3):465-74.
55. Malherbe D C, Kurup D, Wirblich C, Ronk A J, Mire C, al. e. A single dose of replication-competent VSV-vectored vaccine expressing SARS-CoV-2 S1 protects against virus replication in a hamster model of severe COVID-19. npj vaccines. 2021; https://doi.org/10.1038/s41541-021-00352-1.
56. Yahalom-Ronen Y, Tamir H, Melamed S, Politi B, Shifman O, al. e. A single dose of recombinant VSV-AG-spike vaccine provides protection against SARS-CoV-2 challenge. Nature Communications. 2020; 11:6402.
57. O'Donnell K L, Clancy C S, Griffin A J, Shifflett K, Gourdine T, Thomas T, et al. Optimization of single dose VSV-based COVID-19 vaccination in hamsters bioRxiv. 2021; Sep. 3:doi: 10.1101/2021.09.03.458735. Epub Sep. 3, 2021. doi: doi: 10.1101/2021.09.03.458735.
58. Furuyama W, Shifflett K, Pinski A N, Griffin A J, al. e. Rapid protection from COVID-19 in nonhuman primates vaccinated intramuscularly but not intranasally with a single dose of a recombinant vaccine. bioRxiv. 2021:doi: 10.1101/2021.01.19.426885.
59. Ao Z j, Ouyang M J, Olukitibi T A, Yao X J. SARS-CoV-2 Delta Spike Protein Enhances the Viral Fusogenicity and Inflammatory Cytokine Production. bioRxiv. 2021; doi: https://doi.org/10.1101/2021.11.23.469765. doi: doi: https://doi.org/10.1101/2021.11.23.469765.
60. Foster K A, Ostera C G, Mayerb M M, Averya M L, K. L. A. Characterization of the A549 Cell Line as a Type II Pulmonary Epithelial Cell Model for Drug Metabolism. Experimental Cell Research. 1998; 243:359-66.
61. R Yamamoto R, Lin L S L, R., Warren M K, White T J. The human lung fibroblast cell line, MRC-5, produces multiple factors involved with megakaryocytopoiesis. Journal of immunology. 1990; 144:1808-16.
62. Ao Z, Fowke K R, Cohen E A, Yao X. Contribution of the C-terminal tri-lysine regions of human immunodeficiency virus type 1 integrase for efficient reverse transcription and viral DNA nuclear import. Retrovirology. 2005; 2:62. PubMed PMID: 16232319.
63. Jalali N, Brustad H K, Frigessi A, MacDonald E, Meijerink H, Feruglio S, et al. Increased household transmission and immune escape of the SARS-CoV-2 Omicron variant compared to the Delta variant: evidence from Norwegian contact tracing and vaccination data. medRxiv. 2022:2022.02.07.22270437. doi: 10.1101/2022.02.07.22270437.
64. Dogan M, Kozhaya L, Placek L, Gunter C, Yigit M, Hardy R, et al. SARS-CoV-2 specific antibody and neutralization assays reveal the wide range of the humoral immune response to virus. Communications Biology. 2021; 4(1):129. doi: 10.1038/s42003-021-01649-6.
65. Kirola L. Genetic emergence of B.1.617.2 in COVID-19. New Microbes New Infect. 2021; 43:100929. Epub 2021/08/03. doi: 10.1016/j.nmni.2021.100929. PubMed PMID: 34336227; PubMed Central PMCID: PMCPMC8302888.
66. Davis C, Logan N, Tyson G, Orton R, Harvey W T, Perkins J S, et al. Reduced neutralisation of the Delta (B.1.617.2) SARS-CoV-2 variant of concern following vaccination. PLoS pathogens. 2021; 17(12):e1010022. doi: 10.1371/journal.ppat.1010022.
67. Planas D, Veyer D, Baidaliuk A, Staropoli I, Guivel-Benhassine F, Rajah M M, et al. Reduced sensitivity of SARS-CoV-2 variant Delta to antibody neutralization. Nature. 2021; 596(7871):276-80. Epub 2021/07/09. doi: 10.1038/s41586-021-03777-9. PubMed PMID: 34237773.
68. Tian D, Sun Y, Zhou J, Ye Q. The Global Epidemic of the SARS-CoV-2 Delta Variant, Key Spike Mutations and Immune Escape. Frontiers in immunology. 2021; 12. doi: 10.3389/fimmu.2021.751778.
69. Xia X h. Domains and Functions of Spike Protein in Sars-Cov-2 in the Context of Vaccine Design. Viruses. 2021; DOI: 10.3390/v13010109(PMID: 33466921).
70. Dolgin E. COVID vaccine immunity is waning—how much does that matter?Nature. 2021; 597.
71. Azzi L, Dalla Gasperina D, Veronesi G, Shallak M, letto G, lovino D, et al. Mucosal immune response in BNT162b2 COVID-19 vaccine recipients. EBioMedicine. 2022; 75:103788. Epub 2021/12/27. doi: 10.1016/j.ebiom.2021.103788. PubMed PMID: 34954658; PubMed Central PMCID: PMCPMC8718969.
72. de Gier B, Andeweg S, Joosten R, Ter Schegget R, Smorenburg N, van de Kassteele J, et al. Vaccine effectiveness against SARS-CoV-2 transmission and infections among household and other close contacts of confirmed cases, the Netherlands, February to May 2021. Euro Surveill. 2021; 26(31):2100640. doi: 10.2807/1560-7917.ES.2021.26.31.2100640. PubMed PMID: 34355689.
73. Griffin B D, Warner B M, Chan M, Valcourt E, Tailor N, Banadyga L, et al. Host parameters and mode of infection influence outcome in SARS-CoV-2-infected hamsters. iScience. 2021; 24(12):103530. Epub 2021/12/07. doi: 10.1016/j.isci.2021.103530. PubMed PMID: 34870132; PubMed Central PMCID: PMCPMC8627009.
74. Hörner C, Schürmann C, Auste A, Ebenig A, Muraleedharan S, Dinnon K H, et al. A highly immunogenic and effective measles virus-based Th1-biased COVID-19 vaccine. Proceedings of the National Academy of Sciences. 2020; 117(51):32657-66. doi: doi:10.1073/pnas.2014468117.
75. Ewer K J, Barrett J R, Belij-Rammerstorfer S, Sharpe H, Makinson R, Morter R, et al. T cell and antibody responses induced by a single dose of ChAdOx1 nCoV-19 (AZD1222) vaccine in a phase 1/2 clinical trial. Nature Medicine. 2021; 27(2):270-8. doi: 10.1038/s41591-020-01194-5.
76. Morrison C B, Edwards C E, Shaffer K M, Araba K C, Wykoff J A, Williams D R, et al. SARS-CoV-2 infection of airway cells causes intense viral and cell shedding, two spreading mechanisms affected by IL-13. Proceedings of the National Academy of Sciences. 2022; 119(16):e2119680119. doi: doi:10.1073/pnas.2119680119.
77. Zhang J, Xiao T S, Cai Y F, Lavine C L, Peng H Q, Zhu H S, et al. Membrane fusion and immune evasion by the spike protein of SARS-CoV-2 Delta variant. Science. 2021; 374(6573):1353-60.
78. Donofrio G, Franceschi V, Macchi F, Russo L, Rocci A, Marchica V, et al. A Simplified SARS-CoV-2 Pseudovirus Neutralization Assay. Vaccines 2021; 9:389
79. Hu J, Gao Q, He C, Huang A, Tang N, Wang K. Development of cell-based pseudovirus entry assay to identify potential viral entry inhibitors and neutralizing antibodies against SARS-CoV-2. Genes & Diseases 2020; 7:551-7.
80. Flano E, Jewell N A, Durbin R K, Durbin J E. Methods used to study respiratory virus infection. Current protocols in cell biology. 2009; 43:21-6.
81. Gauger P C V, A. L. Serum virus neutralization assay for detection and quantitation of serum-neutralizing antibodies to influenza A virus in swine (Springer, 2014). Animal influenza virus ((Springer). 2014:313-24.
82. Furuyama W, Reynolds P, Haddock E, Meade-White K, Le M Q, Kawaoka Y, et al. A single dose of a vesicular stomatitis virus-based influenza vaccine confers rapid protection against H5 viruses from different clades. NPJ vaccines. 2020; 5(1):1-10.
83. Reed L J, Muench H. A simple method of estimating fifty percent endpoints. American Journal of Epidemiology. 1938; 27(3):493-7

1. A replicative Vesicular stomatitis virus (rVSV) comprising:
a first Filoviridae glycoprotein comprising one or more influenza virus matrix 2 ectodomain peptide inserted into the first Filoviridae glycoprotein; and
a second Filoviridae glycoprotein comprising a SARS-CoV2 Spike protein peptide inserted into the second Filoviridae glycoprotein.

2. The rVSV according to claim 1 wherein the one or more influenza virus matrix 2 ectodomain peptide is inserted into the first Filoviridae glycoprotein in frame such that the one or more influenza virus matrix 2 ectodomain peptide is expressed as a fusion protein with the first Filoviridae glycoprotein.

3. The rVSV according to claim 1 wherein the SARS-CoV2 Spike protein peptide is inserted into the second Filoviridae glycoprotein in frame such that the SAR CoV-2 Spike protein peptide is expressed as a fusion protein with the second Filoviridae glycoprotein.

4. The rVSV according to claim 1 wherein the first Filoviridae glycoprotein is Ebola glycoprotein.

5. The rVSV according to claim 4 wherein the first Filoviridae glycoprotein is a tolerated deletion of the mucin-like domain of the Ebola glycoprotein.

6. The rVSV according to claim 1 wherein the one or more influenza virus matrix 2 ectodomain peptide is inserted in frame in the tolerated deletion of the mucin-like domain of the first Ebola glycoprotein.

7. The rVSV according to claim 6 wherein the influenza virus matrix 2 ectodomain peptide comprises at least 23 consecutive amino acids of the influenza virus matrix 2 ectodomain peptide.

8. The rVSV according to claim 1 wherein the one or more influenza virus matrix 2 ectodomain peptide is selected from: a human influenza virus; an avian influenza virus; a swine influenza virus and combinations thereof.

9. The rVSV according to claim 1 wherein there are two or more influenza virus matrix 2 ectodomain peptides inserted in frame in the tolerated deletion of the mucin-like domain of the first Ebola glycoprotein.

10. The rVSV according to claim 9 wherein each respective one influenza virus matrix 2 ectodomain peptide is separated from a respective adjacent influenza virus matrix 2 ectodomain peptide by a spacer.

11. The rVSV according to claim 9 wherein there are four influenza virus matrix 2 ectodomain peptides inserted in frame in the tolerated deletion of the mucin-like domain of the first Ebola glycoprotein.

12. The rVSV according to claim 11 wherein the four influenza virus matrix 2 ectodomain peptides are two human influenza virus matrix 2 ectodomain peptides, one avian matrix 2 ectodomain peptide and one swine matrix 2 ectodomain peptide.

13. The rVSV according to claim 12 wherein a cassette comprising the four influenza virus matrix 2 ectodomain peptides comprises the amino acid sequence as set forth in SEQ ID NO:6.

14. The rVSV according to claim 1 wherein the Filoviridae virus glycoprotein is the Ebola virus glycoprotein and four copies of the matrix 2 ectodomain peptide are inserted in a tolerated deletion of the mucin-like domain spanning amino acids 305-483 of the native Ebola virus glycoprotein as set forth in SEQ ID NO: 7.

15. The rVSV according to claim 1 wherein the second Filoviridae glycoprotein is Ebola glycoprotein.

16. The rVSV according to claim 15 wherein the second Filoviridae glycoprotein is a tolerated deletion of the mucin domain of the Ebola glycoprotein.

17. The rVSV according to claim 15 wherein the SARS-CoV2 Spike protein peptide is inserted in frame in the tolerated deletion of the mucin-like domain of the second Ebola glycoprotein.

18. The rVSV according to claim 1 wherein the SARS-CoV2 Spike protein peptide is selected from the group consisting of: RBD domain; SPΔCa742 or SPΔS2ΔC.

19. The rVSV according to claim 18 wherein the RBD domain comprises the amino acid sequence as set forth in SEQ ID NO:8.

20. The rVSV according to claim 1 wherein the Filoviridae virus glycoprotein is the Ebola virus glycoprotein and the RBD domain is inserted in a tolerated deletion of the mucin-like domain spanning amino acids 305-483 of the native Ebola virus glycoprotein as set forth in SEQ ID NO: 9.

21. The rVSV according to claim 18 wherein the SPΔCa742 is SARS-CoV2 Delta variant SPΔCa742.

22. The rVSV according to claim 21 wherein the SARS-CoV2 Delta variant SPΔCa742 comprises the amino acid sequence as set forth in SEQ ID NO:10.

23. The rVSV according to claim 18 wherein the SPΔS2ΔC is SARS-CoV2 Delta variant SPΔS2ΔC

24. The rVSV according to claim 23 wherein the SPΔS2ΔC Delta variant comprises the amino acid sequence as set forth in SEQ ID NO:11.

25. (canceled)

26. (canceled)

27. (canceled)

28. A method of eliciting an immune response against an influenza virus matrix 2 ectodomain peptide and/or a SARS-CoV2 Spike protein peptide comprising:
providing a rVSV according to claim 1 and
immunizing an individual in need of immunization against influenza virus matrix 2 ectodomain peptide and/or SARS-CoV2 Spike protein peptide with an effective amount of the rVSV.

29. A method of eliciting an immune response against an influenza virus and/or a SARS-CoV2 comprising:
providing a rVSV according to claim 1 and
immunizing an individual in need of immunization against influenza virus and/or SARS-CoV2 with an effective amount of the rVSV.


SEQUENCE LISTING (17 sequences)

SEQ ID NO: 1 - protein, 23 aa, influenza virus
SLLTEVETPIRNEWGCRCNDSSD

SEQ ID NO: 2 - protein, 23 aa, influenza virus
SLLTEVETPTRNGWECKCSDSSD

SEQ ID NO: 3 - protein, 23 aa, influenza virus
SLLTEVETPIRNEWGCRCNDSSD

SEQ ID NO: 4 - protein, 23 aa, influenza virus
SLLTEVETPIRNGWECRCNDSSD

SEQ ID NO: 5 - protein, 23 aa, artificial
  consensus sequence of 4 M2 domains of influenza viruses
  VARIANT (10)..(10): I or T
  VARIANT (13)..(13): G or E
  VARIANT (15)..(15): G or E
  VARIANT (17)..(17): R or K
  VARIANT (19)..(19): N or S
SLLTEVETPXRNXWXCXCXDSSD

SEQ ID NO: 6 - protein, 107 aa, artificial
  synthetic peptide insert of 4 copies of M2 domain from influenza viruses
  MISC_FEATURE (1)..(3): spacer GGG
  MISC_FEATURE (27)..(29): spacer GGG
  MISC_FEATURE (53)..(55): spacer GGG
  MISC_FEATURE (79)..(81): spacer GGG
  MISC_FEATURE (105)..(107): spacer GGG
GGGSLLTEVETPIRNEWGCRCNDSSDGGGSLLTEVETPTRNGWECKCSDSSDGGGSLLTEVETPIRNEWGCRCNDSSDGGGSLLTEVETPIRNGWECRCNDSSDGGG

SEQ ID NO: 7 - protein, 535 aa, artificial
  ebola glycoprotein mucin like domain deletion with m2 domain 4 insert
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEGPGGGSLLTEVETPIRNEWGCRCNDSSDGGGSLLTEVETPTRNGWECKCSDSSDGGGSLLTEVETPIRNEWGCRCNDSSDGGGSLLTEVETPIRNGWECRCNDSSDGGGSRNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCH

SEQ ID NO: 8 - protein, 195 aa, SARS-CoV2
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 9 - protein, 694 aa, artificial
  Ebola glycoprotein MLD domain deletion, SARS CoV2 RBD insertion
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPSRNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 10 - protein, 1273 aa, SARS CoV2 delta variant
  misc_feature (156)..(157): Xaa can be any naturally occurring amino acid
MFVFLVLLPLVSSQCVNLDTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESXXGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRFRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYACGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 875 aa, artificial
  spike protein construct with internal and terminal deletion
  misc_feature (156)..(157): Xaa can be any naturally occurring amino acid
  MISC_FEATURE (743)..(743): deletion of 381 amino acids
  MISC_FEATURE (876)..(876): deletion of DEDDSEPVLKGVKLHYT from terminus
MFVFLVLLPLVSSQCVNLDTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESXXGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRFRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYACNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKF

SEQ ID NO: 12 - protein, 17 aa, artificial
  C-terminus of spike protein of SARS CoV2
DEDDSEPVLKGVKLHYT

SEQ ID NO: 13 - DNA, 27 bp, artificial
  5′ primer for site directed mutagenesis I742A spike protein
tgtacaatgtatgcatgcggagacagc

SEQ ID NO: 14 - DNA, 27 bp, artificial
  3′ primer for site directed mutagenesis I742A spike protein
gctgtctccgcatgcatacattgtaca

SEQ ID NO: 15 - DNA, 26 bp, artificial
  Sarbeco F1 primer
acaggtacgttaatagttaatagcgt

SEQ ID NO: 16 - DNA, 22 bp, artificial
  Sarbeco R2 primer
atattgcagcagtacgcacaca

SEQ ID NO: 17 - DNA, 26 bp, artificial
  Sarbeco P1 primer
acactagccatccttactgcgcttcg